(19) United States
(12) Patent Application Publication
(10) Pub. No.: US 2025/0020639 A1
(43) Pub. Date: Jan. 16, 2025

(54) ULTRA-BRIGHT NANOPARTICLE FLUORESCENT DYE COMPLEXES

(71) Applicant: CYTEK BIOSCIENCES, INC., Fremont, CA (US)

(72) Inventors: Yu Rong, Fremont, CA (US); Jing Dai, Fremont, CA (US); Xingyong Wu, Fremont, CA (US); Rong Zhang, Fremont, CA (US); Peter Robles, Fremont, CA (US); Su Chen, Fremont, CA (US); Bill Godfrey, Fremont, CA (US); Ming Yan, Fremont, CA (US)

(73) Assignee: CYTEK BIOSCIENCES, INC., Fremont, CA (US)

(21) Appl. No.: 18/747,407

(22) Filed: Jun. 18, 2024

Related U.S. Application Data
(60) Provisional application No. 63/521,692, filed on Jun. 18, 2023.

(51) Int. Cl. G01N 33/533; C09K 11/06; G01N 15/01; G01N 15/10; G01N 15/14; G01N 33/543; G01N 33/569

(52) U.S. Cl. CPC G01N 33/533; C09K 11/06; G01N 15/01; G01N 15/14; G01N 33/54346; G01N 33/56966; G01N 2015/1006

(57) ABSTRACT

In one embodiment, a method to synthesize highly fluorescent complexes is disclosed. The highly fluorescent complexes are synthesized by using inorganic nanoparticles, coupling agents, linkers, and fluorescent dye molecules. The unique nanoparticle-dye complexes (referred to as “SN-dye”) can provide ultra-bright fluorescent labelling. This is demonstrated by coupling the complexes to the antibody and subsequently using the conjugated antibody for more sensitive immunological detection in flow cytometry applications.

### CROSS-REFERENCE TO RELATED APPLICATIONS

This patent application claims the benefit of United States (US) Provisional Patent Application No. 63521692 titled ULTRA-BRIGHT NANOPARTICLE FLUORESCENT DYE COMPLEXES filed on Jun. 18, 2023, by inventors Yu Rong et al., incorporated herein by reference for all intents and purposes.

This patent application is related to United States (US) Non-Provisional patent application Ser. No. 17/304,843 titled METHODS OF FORMING MULTI-COLOR FLUORESCENCE-BASED FLOW CYTOMETRY PANEL filed on Jun. 26, 2021, by inventors Maria Jaimes et al., incorporated herein by reference for all intents and purposes. (US) Non-Provisional patent application Ser. No. 17/304,843 claims the benefit of United States (US) Provisional Patent Application No. 63/045,040 titled METHODS OF FORMING MULTI-COLOR FLUORESCENCE-BASED FLOW CYTOMETRY PANEL filed on Jun. 26, 2020, by inventors Maria Jaimes et al., incorporated herein by reference for all intents and purposes. (US) Non-Provisional patent application Ser. No. 17/304,843 also claims the benefit of United States (US) Provisional Patent Application No. 63/045,103 titled METHODS OF FORMING MULTI-COLOR FLUORESCENCE-BASED FLOW CYTOMETRY PANEL filed on Jun. 27, 2020, by inventors Maria Jaimes et al., incorporated herein by reference for all intents and purposes.

This patent application is further related to United States (US) patent application Ser. No. 15/659,610 titled COMPACT DETECTION MODULE FOR FLOW CYTOMETERS filed on Jul. 25, 2017, by inventors Ming Yan et al., incorporated herein by reference for all intents and purposes. This patent application is further related to U.S. patent application Ser. No. 15/498,397 titled COMPACT MULTI-COLOR FLOW CYTOMETER filed on Apr. 26, 2017, by David Vrane et al. that describes a flow cytometer with which the embodiments can be used and is incorporated herein by reference for all intents and purposes. This patent application is further related to U.S. patent application Ser. No. 16/418,942 titled FAST RECOMPENSATION OF FLOW CYTOMETERY DATA FOR SPILLOVER READJUSTMENTS filed on May 21, 2019, by Zhenyu Zhang that describes matrices with which the embodiments can be used and is incorporated herein by reference for all intents and purposes.

### FIELD

The embodiments of the invention relate generally to methods to synthesize ultra-bright nanoparticle dye complexes for flow cytometry and other biological applications.

### BACKGROUND

Fluorescence detection coupled with bioconjugation techniques is leading to a rapid development in flow cytometry, cell sorting, sequencing, bioimaging, polymerase chain reaction (PCR), fluorescence in situ hybridization (FISH), receptor binding assays, enzyme assays and other advanced fluorescence-based techniques. Organic fluorescent dye molecules, used as probes, were extensively used in the past few decades in these fluorescence techniques. However, organic fluorescent dyes still have limitation due to their low absorptivity, poor photostability, and low brightness, which prevent themselves from further development in high-sensitivity imaging techniques.

Several strategies for enhancing performances of fluorescent dyes have been pursued since they still possess sharp absorption, fluorescence, and other photo-physical properties. For example, SONY Corporation developed a class of fluorescent dyes, by directly incorporating dyes into a polymer backbone using standard phosphoramidite coupling chemistry to form an oligomer. The oligomer maximized the charge density along the polymer backbone that would rigidify through charge repulsion and thus allow for separation of two fluorophores attached to either end of a “dumbbell” sequence. (See “A Novel Class Of Polymeric Fluorescent Dyes Assembled Using A DNA Synthesizer” by Tracy MatrayI, Sharat Singh, Hesham Sherif, Kenneth Farber, Erin Kwang, Michael VanBrunt, Eriko Matsui, Hiroaki Yada; PLOS ONE journal; Dec. 4, 2020; vol. 15(12), pages 1-13).

Another group of researchers describe developing bright fluorophores in U.S. Patent Application Publication No. 2018/0296705A1. These bright fluorophores use boron nitride nano tubes (BNNTs) as a carrier to load more fluorescent dyes on it to enhance brightness. (See U.S. patent application Ser. No. 15/953,200, filed on Apr. 13, 2018, by Yoke Khin Yap et al., incorporated herein by reference for all intents and purposes).

Another group of researchers, led by Igor Sokolov, developed fluorescent doped nanoparticles (NPs). In the research lead by Igor Sokolov, the fluorescent doped nanoparticles exhibited improved brightness and photostability as compared to single fluorescent molecules. The fluorescent doped nanoparticles had multiple dye molecules per particle and a protective latex matrix. (See “Ultrabright Fluorescent Silica Nanoparticles For In Vivo Targeting Of Xenografted Human Tumors And Cancer Cells In Zebrafish” by Saquib Ahmed M. A. Peerzade, Xiaodan Qin, Fabrice J. F. Laroche, Shajesh Palantavida, Maxim Dokukin, Berney Peng, Hui Feng and Igor Sokolov, Nanoscale, Journal of The Royal Society of Chemistry, November 2019, pages 22316-22327, incorporated herein by reference for all intents and purposes).

However, some of these prior techniques have large and irregular size, resulting in strong fluorescence background noise in biological applications. Some of these prior techniques are difficult to quantitatively calculate loading ratio of dyes, resulting in poor reproducible performance. Some of these prior techniques have complicated synthetic processes. Therefore, there is a need for a new type of fluorescent dye system with very high brightness and good reproducible performance.

### BRIEF SUMMARY

The embodiments are generally summarized by the claims that follow below.

In some aspects, the techniques described herein relate to a method for producing conjugated inorganic nanoparticle fluorescent dye complexes for flow cytometry and biological applications, the method including: a) covering a surface of a plurality of inorganic nanoparticles with a functional group R using a coupling agent to form a plurality of functionalized inorganic nanoparticles; b) coupling fluorescent dyes to the plurality of functionalized inorganic nanoparticles to form a plurality of functionalized fluorescent inorganic nanoparticles; c) coupling a plurality of first linker molecules, attached to a functional group R″, to the plurality of fluorescent functionalized inorganic nanoparticles; and d) bioconjugating the plurality of functionalized fluorescent inorganic nanoparticles with antibodies or other bioactive molecules.

In some aspects, the techniques described herein relate to a method, wherein using the coupling agent to cover the surface of the plurality of inorganic nanoparticles with functional groups further includes, hydrolyzing the coupling agent.

In some aspects, the techniques described herein relate to a method, wherein coupling fluorescent dyes to the functionalized inorganic nanoparticles further includes, mixing fluorescent dyes attached to the first functional group with functionalized inorganic nanoparticles.

In some aspects, the techniques described herein relate to a method, wherein coupling a plurality of first linker molecules to the surface of the plurality of functionalized inorganic nanoparticles further includes, mixing the plurality of first linker molecules, attached to a functional group R′ and a functional group R″, with the functionalized inorganic nanoparticles, wherein the functional group R′ reacts with the functional group R covering the surface of a plurality of inorganic nanoparticles.

In some aspects, the techniques described herein relate to a method, further includes, changing the functional group R″, attached to the plurality of first linker molecules, to a functional group R2, by mixing a plurality of second linker molecules to the fluorescent functionalized inorganic nanoparticles, wherein a functional group of the plurality of second linker molecules reacts with the functional group R″ of the first plurality of linker molecules to form the functional group R2;

In some aspects, the techniques described herein relate to a method, further including reacting linker molecules or oligomers with a plurality of antibodies or other bioactive molecules to activate the plurality of antibodies or other bioactive molecules.

In some aspects, the techniques described herein relate to a method, further including directly bio-conjugating functionalized fluorescent inorganic nanoparticles with activated antibodies or other bioactive molecules

In some aspects, the techniques described herein relate to a method, wherein: the inorganic nanoparticles are metal oxide nanoparticles,

In some aspects, the techniques described herein relate to a method, wherein: the inorganic nanoparticles include alumina nanoparticles, silica nanoparticles, titania nanoparticles, indium tin oxide nanoparticles, zinc oxide nanoparticles, iron oxide nanoparticles, antimony tin oxide nanoparticles, or nanoparticles covered with an inorganic metal oxide layer.

In some aspects, the techniques described herein relate to a method, wherein: the size of the inorganic nanoparticles is less than 500 nanometers, 200 nanometers, 100 nanometers, 50 nanometers, 25 nanometers, 15 nanometers, 10 nanometers, or 5 nanometers.

In some aspects, the techniques described herein relate to a method, wherein: the coupling agent is one of a silane coupling agent, a titanate coupling agent, an aluminate coupling agent, a zirconate coupling agent, a phosphate coupling agent, and a borate coupling agent.

In some aspects, the techniques described herein relate to a method, wherein: the functional organic groups on the coupling agents include one or more of alkylhalide, azide, amino, alkyne, aldehyde, maleimide, hydroxyl, acetal, isocyanate, epoxide, acrylate, sulfonate (tosyl, mesyl), nitrophenyl carbonate, Biotins, folic acid, methacrylate, mercapto, tetrafluorophenyl esters, succinimidyl ester, pentafluorophenyl ester, hydrazides, vinyl, vinylsulfone, dibenzocyclooctyne group (DBCO), and methyltetrazine.

In some aspects, the techniques described herein relate to a method, wherein: the linker that connects the functional groups to the silicon atom includes one of an alkyl chain, a peptide chain, and a polyethylene oxide chain.

In some aspects, the techniques described herein relate to a method, wherein: the number of repeated units of the linker ranges from 5,000 to 1, from 3,000 to 1, from 2,000 to 1, from 1,000 to 1, from 500 to 1, from 100 to 1, or from 20 to 1.

In some aspects, the techniques described herein relate to a method, wherein: the fluorescent dye is a fluorescent chemical compound that can emit light upon laser excitation.

In some aspects, the techniques described herein relate to a method, wherein: the fluorescent dye is at least one of BODIPY derivatives, dipyrrin-metal derivatives, Atto derivatives, Cyanine derivatives, squaraine derivatives, Fluorescein derivatives, porphyrin, metalloporphyrin derivatives, phthalocyanine derivatives, Rhodamine derivatives, lanthanide complexes derivatives, and Pyrene dyes.

In some aspects, the techniques described herein relate to a method, wherein: the fluorescent dye is an organic fluorescent dye with a narrow bandwidth of light absorption between 260 nanometers and 900 nanometers and a narrow bandwidth of fluorescence between 260 nanometers and 1100 nanometers.

In some aspects, the techniques described herein relate to a method, wherein: the fluorescent dye has functional groups that can react with functional groups on a surface of inorganic nanoparticles.

In some aspects, the techniques described herein relate to a method, wherein: the functional groups on the fluorescent dye is at least one of an amino, an alkylhalide, an azide, an alkyne, an aldehyde, a maleimide, a hydroxyl, an acetal, an isocyanate, an epoxide, an acrylate, a sulfonate (tosyl, mesyl), a nitrophenyl carbonate, a Biotins, a folic acid, a methacrylate, a mercapto, a tetrafluorophenyl ester, a succinimidyl ester, a pentafluorophenyl ester, a hydrazides, a vinyl, a vinylsulfone, a dibenzocyclooctyne group (DBCO), and a methyltetrazine and other reactive functional organic groups.

In some aspects, the techniques described herein relate to a method, wherein: the functional groups on the fluorescent dye are reactive functional organic groups.

In some aspects, the techniques described herein relate to a method, wherein: the fluorescent dyes react with functional groups on surface of inorganic nanoparticle by a condensation reaction, a click chemistry reaction, a photochemistry reaction, a Suzuki coupling reaction, a Stille coupling reaction, a Sonogashira coupling reaction; or a Heck, Mcmurray and Knoevenagel, Wittig, Horner reaction.

In some aspects, the techniques described herein relate to a method, wherein: the linker molecules or oligomers with functional groups are an oligomer chain with one functional group, an oligomer chain with two functional groups, or branched oligomers with multi-functional groups.

In some aspects, the techniques described herein relate to a method, wherein: the functional groups in linker molecules or oligomers include one or more of an amino, an alkylhalide, an azide, an alkyne, an aldehyde, a maleimide, a hydroxyl, an acetal, an isocyanate, an epoxide, an acrylate, a sulfonate (toys, mesyl), a nitrophenyl carbonate, a Biotins, a folic acid, a methacrylate, a mercapto, a tetrafluorophenyl ester, a succinimidyl ester, a pentafluorophenyl ester, a hydrazides, a vinyl, a vinylsulfone, a dibenzocyclooctyne group (DBCO), an a methyltetrazine.

In some aspects, the techniques described herein relate to a method, wherein: the functional groups in linker molecules or oligomers are reactive functional organic groups.

In some aspects, the techniques described herein relate to a method, wherein: the backbone of the linker molecules are one of an alkyl chain, a peptide chain, and a polyethylene oxide chain.

In some aspects, the techniques described herein relate to a method, wherein: the number of repeated units in the linker molecules range from 10,000 to 1, from 5,000 to 1, from 3,000 to 1, from 2,000 to 1, from 1,000 to 1, from 500 to 1, from 100 to 1, or from 20 to 1.

In some aspects, the techniques described herein relate to a reagent kit for analysis of blood cells by a spectral flow cytometer, the reagent kit including: one or more test tubes having one or more reagents of the reagent composition of an antibody conjugated to an inorganic nanoparticle fluorescent dye complex; wherein the inorganic nanoparticle fluorescent dye complex further includes, an inorganic nanoparticle coupled to; one or more fluorescent dyes; and one or more linker molecules coupling the inorganic nanoparticle to the antibody.

### BRIEF DESCRIPTIONS OF THE DRAWINGS

This patent or application file contains at least one drawing executed in color. Copies of this patent or patent application publication with color drawing(s) will be provided by the United States Patent and Trademark Office upon request and payment of the necessary fee.

These drawings and other features, aspects will become better understood with regards to the following description, appended claims, and accompanying drawings where:

FIG. 1 shows a simple schematic structure of a nanoparticle-dye complex (SN-dye) bio-conjugated with an antibody.

FIGS. 2A-2E show schematics of the synthesis of nanoparticle-dye complexes (SN-dye) and bioconjugation with an antibody.

FIG. 3 shows a general formula for a silane coupling agent.

FIG. 4 shows examples of silane coupling agents.

FIG. 5 shows a mechanism of hydrolysis of silane coupling agents and formation of a functional layer on the surface of inorganic nanoparticles.

FIG. 6 shows hydrolysis of different silane coupling agents with different functional groups and formation of functional layer on surface of inorganic nanoparticles.

FIGS. 7A-7D show general formula for a linker molecule.

FIGS. 8A-8C illustrate examples of linker molecules.

FIGS. 9A-9D illustrate synthesis of nanoparticle-cFluor V420 complexes (SN-cFluor V420) and bioconjugation with an antibody.

FIG. 10A illustrate a density plot of the side scanner channel (SSC) versus the violet channel V2 for whole blood cells stained with CD4-SN-cFluor V420.

FIG. 10B illustrate a density plot of the side scanner channel (SSC) versus the violet channel V2 for whole blood cells stained with CD4-cFluor V420.

FIG. 11A illustrates a full spectral signature of CD4-SN-cFluor V420 with multi-laser excitation.

FIG. 11B illustrates a full spectral signature of CD4-cFluor V420 with multi-laser excitation.

FIG. 12 illustrates plots of full spectrum flow cytometry intensity distributions of whole blood cell labeled using CD4-cFluor V420 and CD4-SN-cFluor V420 and excited by multiple lasers (3 to 5) including a violet laser.

FIGS. 13A-13E illustrate synthesis of nanoparticle-cFluorUV388 complexes (SN-cFluor UV388) and bioconjugation with an antibody.

FIG. 14A illustrates a density plot of the side scanner channel (SSC) versus ultra-violet channel UV2 for whole blood cells stained with CD4-cFluorUV388.

FIG. 14B illustrates a density plot of the side scanner channel (SSC) vs. ultra-violet channel UV2 for whole blood cells stained with CD4-SN-cFluorUV388.

FIG. 15A illustrates a full spectral signature of a CD4-cFluor UV388 with multi-laser excitation.

FIG. 15B illustrates a full spectral signature of a CD4-SN-cFluor UV388 with multi-laser excitation.

FIG. 16 illustrates plots of full spectrum flow cytometry intensity distributions of whole blood cells labeled using CD4-cFluor UV388 and CD4-SN-cFluor UV388.

FIGS. 17A-17E illustrate synthesis of nanoparticle-cFluor B532 complexes (SN-cFluor B532) and bioconjugation with antibody.

FIG. 18A illustrates a density plot of the side scanner channel (SSC) versus. blue channel B2 for whole blood cells stained with CD4-cFluor B532.

FIG. 18B. illustrates a density plot of the side scanner channel (SSC) versus blue channel B2 for whole blood cells stained with CD4-SN-cFluor B532.

FIG. 19A illustrates a full spectral signature of a CD4-cFluor B532 with multi-laser excitation.

FIG. 19B illustrates a full spectral signature of a CD4-SN-cFluor B532 with multi-laser excitation.

FIG. 20 illustrates plots of. full spectrum flow cytometry intensity distributions of whole blood cell labeled using CD4-cFluor B532 and CD4-SN-cFluor B532.

FIGS. 21A-21E illustrate synthesis of nanoparticle-cFluor R780 complexes (SN-cFluor R780 and bioconjugation with antibody.

FIG. 22A illustrates a density plot of the side scanner channel (SSC) versus red channel R7 for whole blood cells stained with CD4-SN-cFluor R780.

FIG. 22B illustrates a density plot of the side scanner channel (SSC) versus red channel R7 for whole blood cells stained with CD4-cFluor R780.

FIG. 23A illustrates a plot of a full spectral signature of a CD4-SN-cFluor R780 with multi-laser excitation.

FIG. 23B illustrates a plot of a full spectral signature of a CD4-cFluor R780 with multi-laser excitation.

FIG. 24 illustrates plots of full spectrum flow cytometry intensity distributions of whole blood cell labeled using CD4-cFluor R780 and CD4-SN-cFluor R780.

FIG. 25 is a basic conceptual diagram of a flow cytometer system.

FIG. 26 is a conceptual diagram of a fluorochrome, an antibody, and a cell.

FIG. 27 is a conceptual diagram of forming a reference sample with a bead.

FIG. 28A is an overall method for performing an experiment with a biological sample and/or running calibration beads through a flow cytometer.

FIG. 28B is a diagram of a calibrating process of a flow cytometer with single stained compensation controls to generate an initial spillover matrix or reference matrix with levels of compensation.

FIG. 28C is a diagram of running a sample through the flow cytometer resulting in a mixed sample event vector with an overlapping spectral profile due to multi-stained cells or particles.

FIG. 28D is a diagram of a processing using an inverse matrix (determined from the initial spillover matrix and/or the initial reference matrix with fine adjustments) on the event data to generate a compensated sample event vector or an unmixed sample event vector.

FIGS. 28E (28E-1 and 28E-2) is a schematic diagram of a full spectrum flow cytometer.

FIG. 28F shows configuration details of the photo detectors in the detector modules for a full spectrum flow cytometer.

FIGS. 28G (28G-1 and 28G-2) illustrates the individual spectrum signature of each color laser and combined full spectrum signature of an exemplary fluorochrome.

FIG. 29 is a listing of the exemplary cell markers and fluorochromes in a 28 color Optimized Multicolor Immunofluorescence Panel (OMIP).

FIG. 30A illustrates the spectrum signature of BUV737.

FIG. 30B illustrates the spectrum signature of BV421.

FIG. 31 is a top view of an optical plate assembly in a modular flow cytometry system with three excitation lasers.

FIG. 32 is a top view of an optical plate assembly in a modular flow cytometry system with five excitation lasers, including a UV excitation laser, of the full spectrum flow cytometer.

FIG. 33 illustrates a plurality of configurations for the modular flow cytometer.

FIGS. 34A-34B are block diagrams of a computer system that can execute software instructions to display a graphical user interface and remotely interact with a web-based spectrum viewer software application.

### DETAILED DESCRIPTION

In the following detailed description of the disclosed embodiments, numerous specific details are set forth in order to provide a thorough understanding. However, it will be obvious to one skilled in the art that the disclosed embodiments may be practiced without these specific details. In other instances, well known methods, procedures, components, and subsystems have not been described in detail so as not to unnecessarily obscure aspects of the disclosed embodiments.

The disclosed embodiments include methods, apparatus, systems, and compositions of matter for nanoparticle fluorescent dye complexes. The nanoparticle fluorescent dye complex has a very high brightness, and its synthesis is disclosed herein.

Generally, a nanoparticle (NP) is a small particle that ranges in size between 1 nanometer (nm) to 100 nanometers in size. Undetectable by the human eye, nanoparticles can exhibit significantly different physical and chemical properties to their larger material counterparts. Material properties change as the size of the material object approaches the atomic scale. This is due to the surface area to volume ratio increasing, resulting in the material's surface atoms dominating the material performance. Owing to their very small size, nanoparticles have a very large surface area to volume ratio when compared to bulk material, such as powders, plate and sheet. This feature enables nanoparticles to possess unexpected optical, physical and chemical properties, as they are small enough to confine their electrons and produce quantum effects. [See https://www.twi-global.com/technical-knowledge/faqs/what-are-nanoparticles].

Nanoparticles can be formed from organic or inorganic material. Nanoparticles formed from inorganic material can be referred to inorganic nanoparticles. Nano-sized inorganic particles (inorganic nanoparticles) of either simple or complex nature can display unique, physical and chemical properties and represent an increasingly important material in the development of novel nanodevices which can be used in numerous physical, biological, biomedical and pharmaceutical applications.

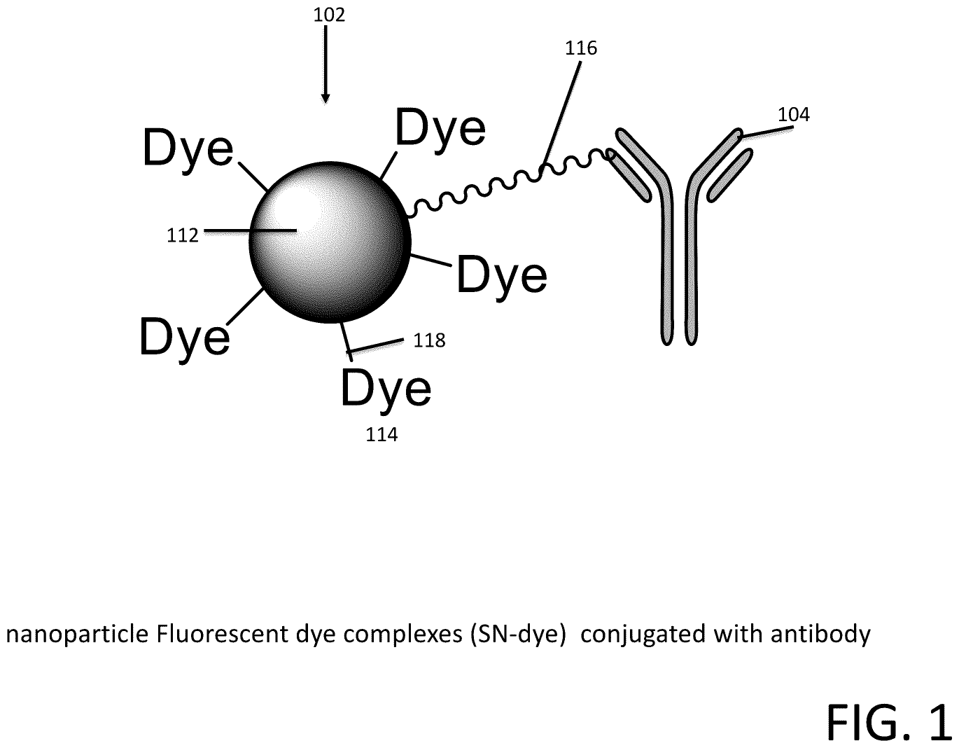

FIG. 1 illustrates a nanoparticle fluorescent dye complex 102 bio-conjugated with an antibody 104. The nanoparticle fluorescent dye complex 102 includes an inorganic nanoparticle 112, one or more fluorescent dye molecules (dye) 114, and one or more linker molecules 116 with two functional groups (R′, R2 or R″). A coupling agent 118 with at least one functional group couples each of the one or more fluorescent dye molecules 114 to the inorganic nanoparticle 112. While one nanoparticle fluorescent dye complex 102 and one antibody 104 are often shown in the figures for description, a sample solution will have a plurality of each that are run through a flow cytometer to obtain a count, a measure of intensity over wavelengths of each detector channel to form a spectral signature.

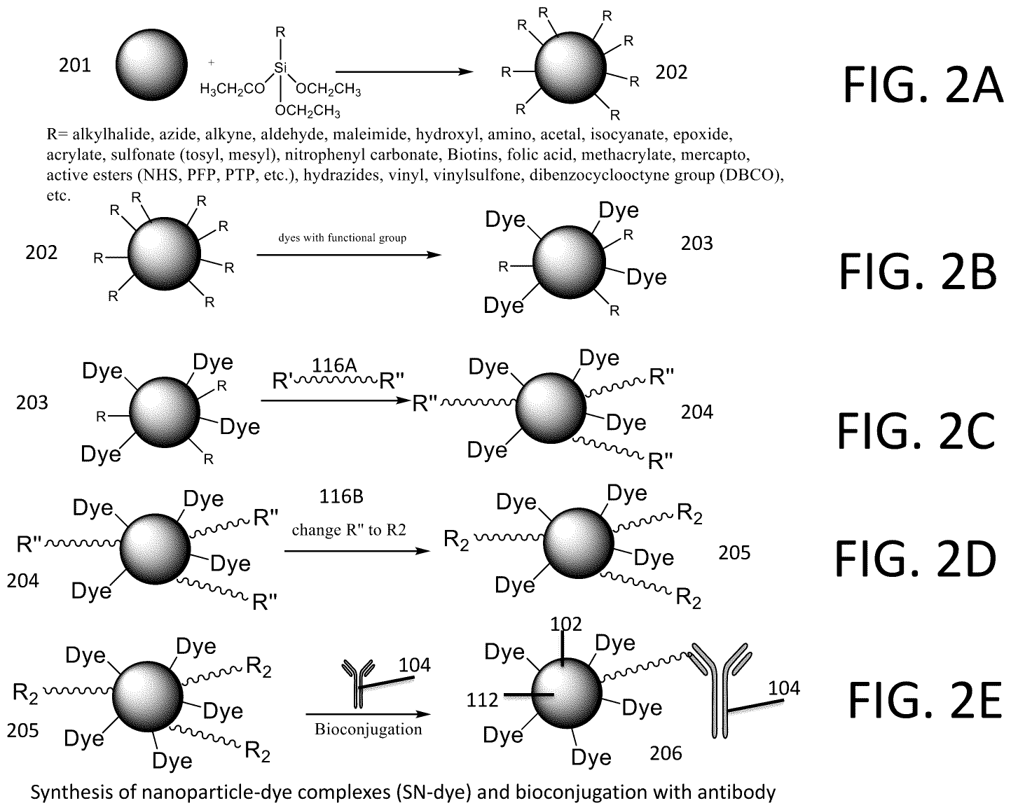

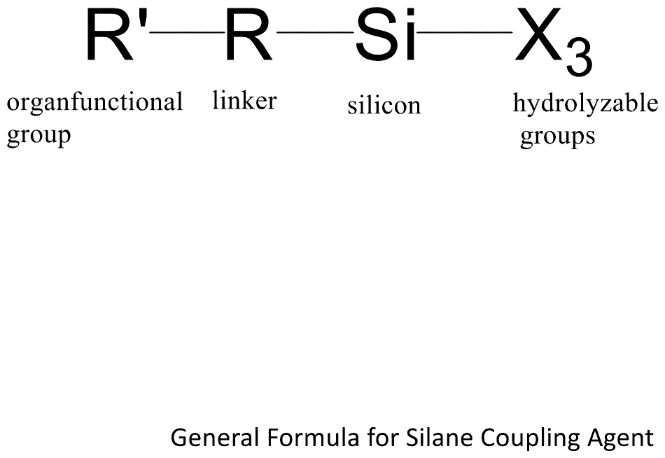

FIGS. 2A-2E illustrate the preparation of a nanoparticle fluorescent dye complex 102 and its conjugation with the antibody 104. In FIG. 2A, a prepared inorganic nanoparticle 201 is initially covered with a functional group R using a coupling agent, thereby forming a functionalized inorganic nanoparticle 202. The functional group R can react with fluorescent dyes as shown in FIG. 2B to couple the dyes to the functionalized inorganic nanoparticle 202 to form the fluorescent functionalized inorganic nanoparticle 203. In FIG. 2C, first linker molecules 116A, attached to functional group R′ and functional group R″, react with the functional group R on the fluorescent functionalized inorganic nanoparticle 203 to form the functional group R″ on a fluorescent inorganic nanoparticle 204. It is functionalized on the linker molecule instead of on the surface of the inorganic nanoparticle. In FIG. 2D, a second linker molecule 116B reacts with the functional group R″ on the inorganic nanoparticle 204 to provide the functional group R2 thereon. In FIG. 2E, one of the functional groups R2 on the inorganic nanoparticle 205, further bioconjugates (directly bio-conjugating) with an antibody 104, or other bio-active molecules to form a nanoparticle-dye complex 206 conjugated with the antibody 104.

In some embodiments, the inorganic nanoparticles are nanoparticles made of metal oxide, on which there are hydroxyl groups. Examples of inorganic nanoparticles include but are not limited to alumina nanoparticles, silica nanoparticles, titania nanoparticles, indium tin oxide nanoparticles, zinc oxide nanoparticles, iron oxide nanoparticles, antimony tin oxide nanoparticles, or nanoparticles covered with inorganic metal oxide layer, etc.

The size (diameter) of inorganic nanoparticles can vary. In some embodiments, the size of inorganic nanoparticles is less than 500 nanometers (nm). In some embodiments, the size of inorganic nanoparticles is less than 300 nm. In some embodiments, the size of inorganic nanoparticles is less than 200 nm. In some embodiments, the size of inorganic nanoparticles is less than 100 nm. In some embodiments, the size of inorganic nanoparticles is less than 50 nm. In some embodiments, the size of inorganic nanoparticles is less than 25 nm. In some embodiments, the size of inorganic nanoparticles is less than 15 nm. In some embodiments, the size of inorganic nanoparticles is less than 10 nm. In some embodiments, the size of inorganic nanoparticles is less than 5 nm.

In some embodiments, coupling agents are used to form the nanoparticles because of their ability to incorporate an organic-compatible functionality and an inorganic-compatible functionality within the same molecule. Organic functionality, can include but are not limited to amino, alkylhalide, azide, alkyne, aldehyde, maleimide, hydroxyl, acetal, isocyanate, epoxide, acrylate, sulfonate (tosyl, mesyl), nitrophenyl carbonate, Biotins, folic acid, methacrylate, mercapto, tetrafluorophenyl esters, succinimidyl ester, pentafluorophenyl ester, hydrazides, vinyl, vinylsulfone, dibenzocyclooctyne group (DBCO), methyltetrazine and other functional organic groups. These functional organic groups can be readily incorporated into a coupling agent.

Referring now to FIG. 3, a general formula of a silane coupling agent is shown. Si represents a silicon atom. X3 represents hydrolysable groups. The R′ represents an organofunctional group. The R group in formula represents a linker.

The organofunctional group R′ can be an amino, alkylhalide, azide, alkyne, aldehyde, maleimide, hydroxyl, acetal, isocyanate, epoxide, acrylate, sulfonate (tosyl, mesyl), nitrophenyl carbonate, Biotins, folic acid, methacrylate, mercapto, tetrafluorophenyl esters, succinimidyl ester, pentafluorophenyl ester, hydrazides, vinyl, vinylsulfone, dibenzocyclooctyne group (DBCO), methyltetrazine or other functional organic groups.

The linker, the R group, may include but not limited to alkyl chain, peptide chain, polyethylene oxide chain, etc. The number of repeated units in linker could be from 1 to 5,000. In some embodiments, the number of repeated units in linker may be from 1 to 3000. In some embodiments, the number of repeated units in linker may be from 1 to 1000. In some embodiments, the number of repeated units in linker may be from 1 to 500. In some embodiments, the number of repeated units in linker may be from 1 to 200. In some embodiments, the number of repeated units in linker may range from 1 to 50. In some embodiments, the number of repeated units in linker may be from 1 to 20. X represents hydrolysable groups, it can be methoxyl, ethoxyl, acetoxy, etc.

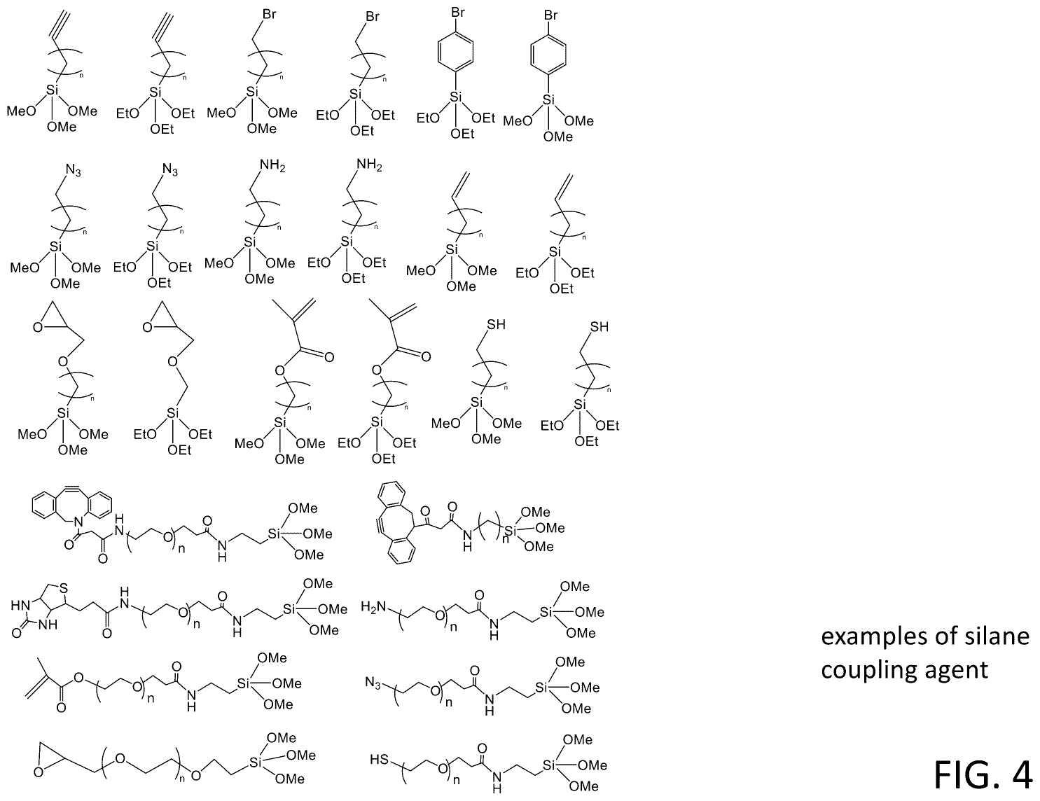

FIG. 4 shows some examples of silane coupling agents. In some embodiments, silane coupling agents include but not limited to vinyltrimethoxysilane, vinyltriethoxysilane, glycidoxypropyl trimethoxysilane, glycidoxypropyl triethoxysilane, p-styryltrimethoxysilane, p-styryltriethoxysilane, 3-methacryloxypropyl trimethoxysilane, 3-methacryloxypropyl triethoxysilane, 8-methacryloxyoctyl triethoxysilane, 8-methacryloxyoctyl trimethoxysilane, 3-acryloxypropyl trimethoxysilane, 3-aminopropyl trimethoxysilane, 3-aminopropyl triethoxysilane, 3-ureidopropyl trimethoxysilane, 3-ureidopropyl triethoxysilane, 3-isocyanatepropyl trimethoxysilane, 3-isocyanatepropyl triethoxysilane, tris-(trimethoxysilylpropyl)-isocyanurate, 3-isocyanatepropyl triethoxysilane, tris-(triethoxysilylpropyl)-isocyanurate, 3-mercaptopropyltrimethoxysilane, 3-mercaptopropyltriethoxysilane, 3-(trimethoxysilyl)propylsuccinic anhydride, 3-(triethoxysilyl)propylsuccinic anhydride, chloropropyltrimethoxysilane, (3-azidopropyl) trimethoxysilane, etc.

In some embodiments, the coupling agents with functional groups include but not limited to silane coupling agent, titanate coupling agent, aluminate coupling agent, zirconate coupling agent, phosphate coupling agent, borate coupling agent, etc.

In some embodiments, the titanate coupling agent, the aluminate coupling agent, the zirconate coupling agent, the phosphate coupling agent, and the borate coupling agent may also be used for grafting functional group on surface of inorganic nanoparticles since they have more functional sites relative to the di-functional silane.

In some embodiments, silane coupling agents that contain three inorganic reactive groups on silicon (usually methoxy, ethoxy or acetoxy) will bond well to the hydroxyl groups on inorganic nanoparticles. The alkoxy groups on silicon hydrolyze to silanols, either through the addition of water or from residual water on the inorganic surface. Then the silanols coordinate with hydroxyl groups on the inorganic surface to form an oxane bond and eliminate water.

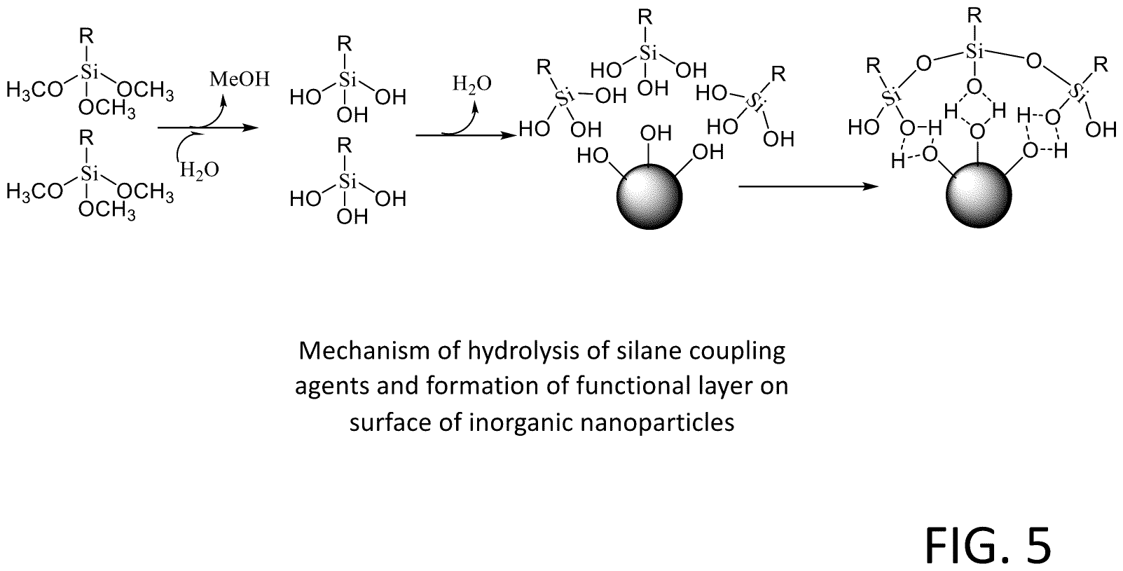

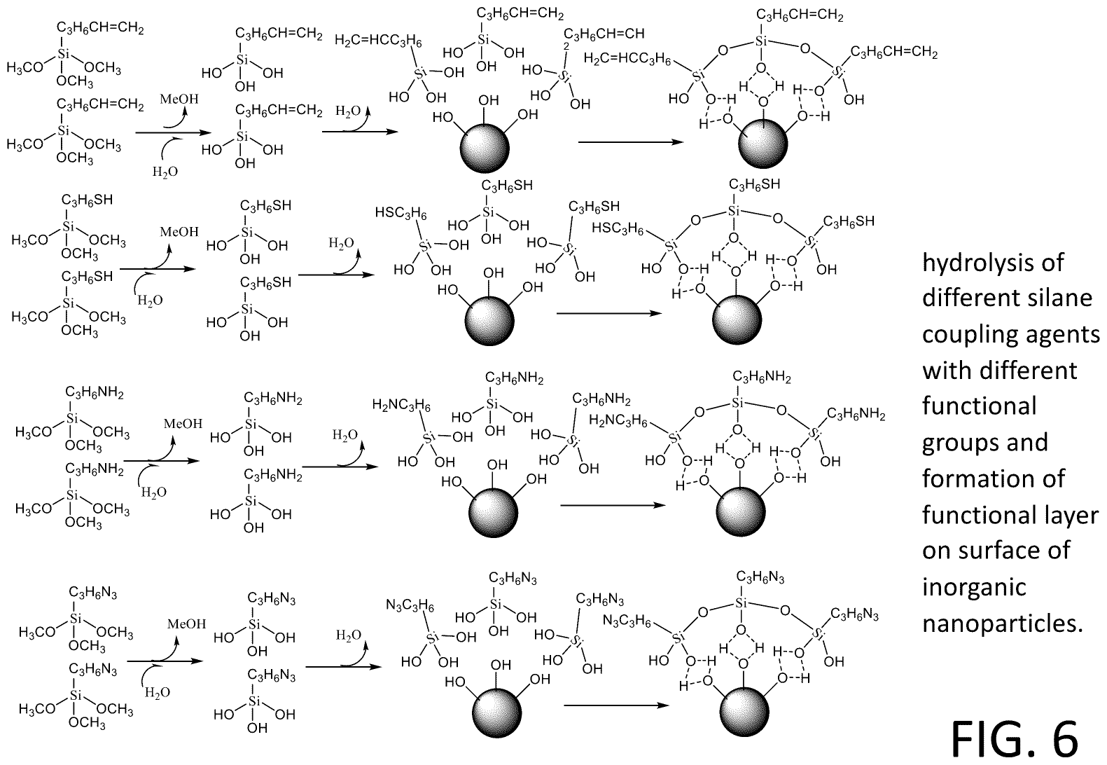

FIG. 5 illustrates hydrolysis of several silane coupling agents and the formation of a functional layer on the surface of the inorganic nanoparticles. FIG. 6 illustrates hydrolysis of several silane coupling agents with different functional groups and further the formation of a functional layer on the surface of the inorganic nanoparticles.

In some embodiments, a fluorescent dye is a fluorescent chemical compound that can emit light upon laser excitation. Fluorophores typically contain several combined aromatic groups, or planar or cyclic molecules with several bonds. Fluorescent dyes include but not limited to BODIPY derivatives, dipyrrin-metal derivatives, ATTO derivatives, Cyanine derivatives, squaraine derivatives, Fluorescein derivatives, porphyrin, metalloporphyrin derivatives, phthalocyanine derivatives, Rhodamine derivatives, lanthanide complexes derivatives, Pyrene dyes, and other organic fluorescent dyes with a narrow bandwidth of light absorption between 260 nm and 900 nm and narrow bandwidth fluorescence between 260 nm and 1100 nm.

In some embodiments, fluorescent dye contains functional group which can react with functional groups on the surface of inorganic nanoparticles. Functional groups on fluorescent dye include but are not limited to amino, alkylhalide, azide, alkyne, aldehyde, maleimide, hydroxyl, acetal, isocyanate, epoxide, acrylate, sulfonate (tosyl, mesyl), nitrophenyl carbonate, Biotins, folic acid, methacrylate, mercapto, tetrafluorophenyl esters, succinimidyl ester, pentafluorophenyl ester, hydrazides, vinyl, vinylsulfone, dibenzocyclooctyne group (DBCO), methyltetrazine and other functional organic groups.

In some embodiments, fluorescent dyes can react with functional groups on the surface of inorganic nanoparticle via condensation reaction, click chemistry reaction, photochemistry reaction, Suzuki coupling reaction, Stille coupling reaction, Sonogashira coupling reaction, Heck, Mcmurray and Knoevenagel, Wittig, Horner reaction, etc.

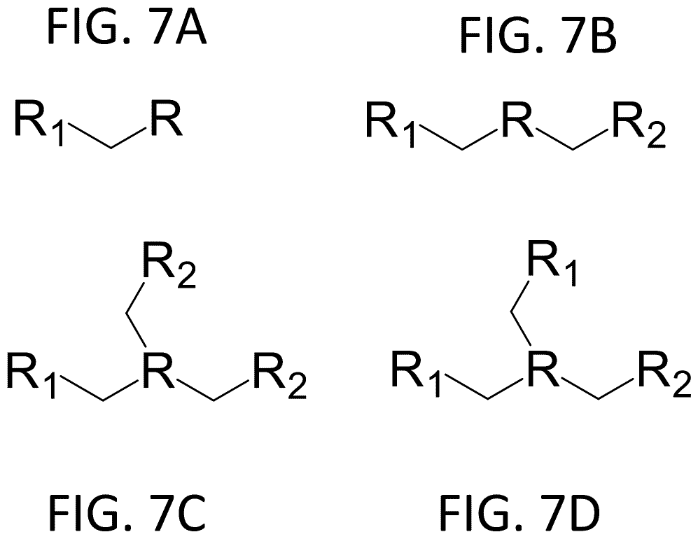

In some embodiments, linker molecules are oligomers with functional groups. FIGS. 7A-7D illustrate general formulas for linker molecules that can be used. As shown in FIG. 7A, the general formula for a linker molecule can be an oligomer chain with one functional group R1. As shown in FIG. 7B, the general formula for a linker molecule can be an oligomer chain with two functional groups R1,R2, As shown in FIG. 7C, the general formula for a linker molecule can be a branched oligomers with multi-functional groups R1,R2, in which the functional group R1 can react with the antibody, or other bioactive molecular, and the functional group R2 can further react with functional groups on the surface of inorganic nanoparticle. Backbone of linker molecules R can include but is not limited to alkyl chain, peptide chain, polyethylene oxide chain, etc.

The number of repeated units in linker molecules can be within various ranges based on concentration. In some embodiments, the number of repeated units in linker molecules can range from 1 to 10,000. In some embodiments, the number of repeated units in linker molecules can range from 1 to 5,000. In some embodiments, the number of repeated units in linker molecules can range from 1 to 4,000. In some embodiments, the number of repeated units in linker molecules can range from 1 to 2,000. In some embodiments, the number of repeated units in linker molecules can range from 1 to 1,000. In some embodiments, the number of repeated units in linker molecules can range from 1 to 500. In some embodiments, the number of repeated units in linker molecules can range from 1 to 200. In some embodiments, the number of repeated units in linker molecules can range from 1 to 100. In some embodiments, the number of repeated units in linker molecules can range from 1 to 50.

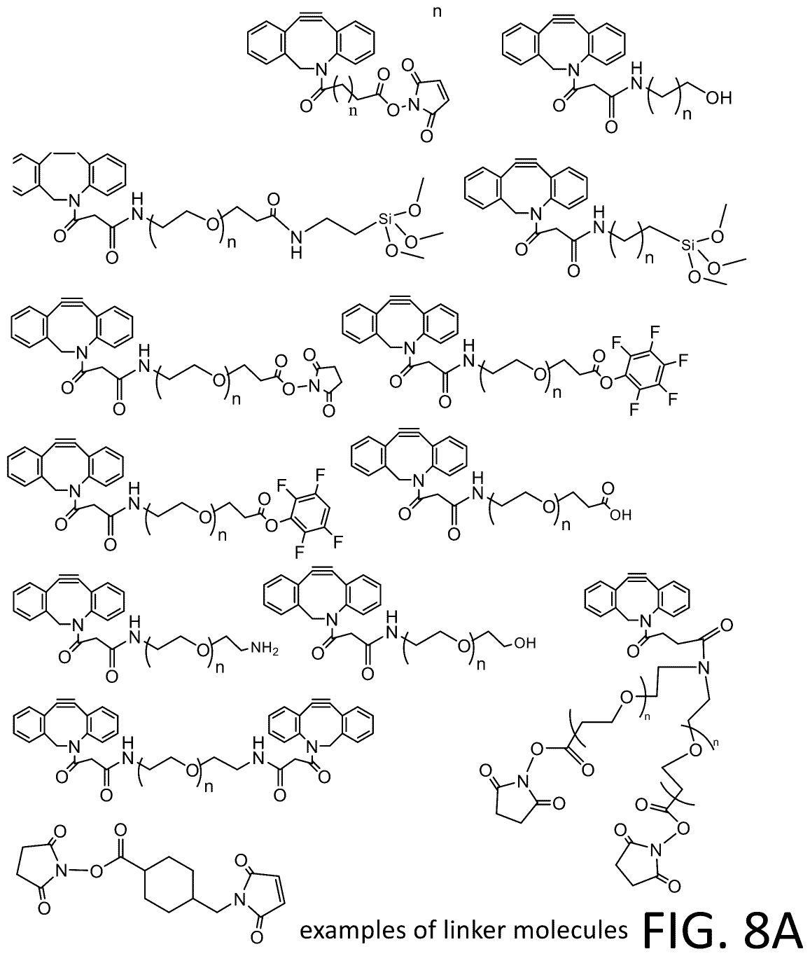

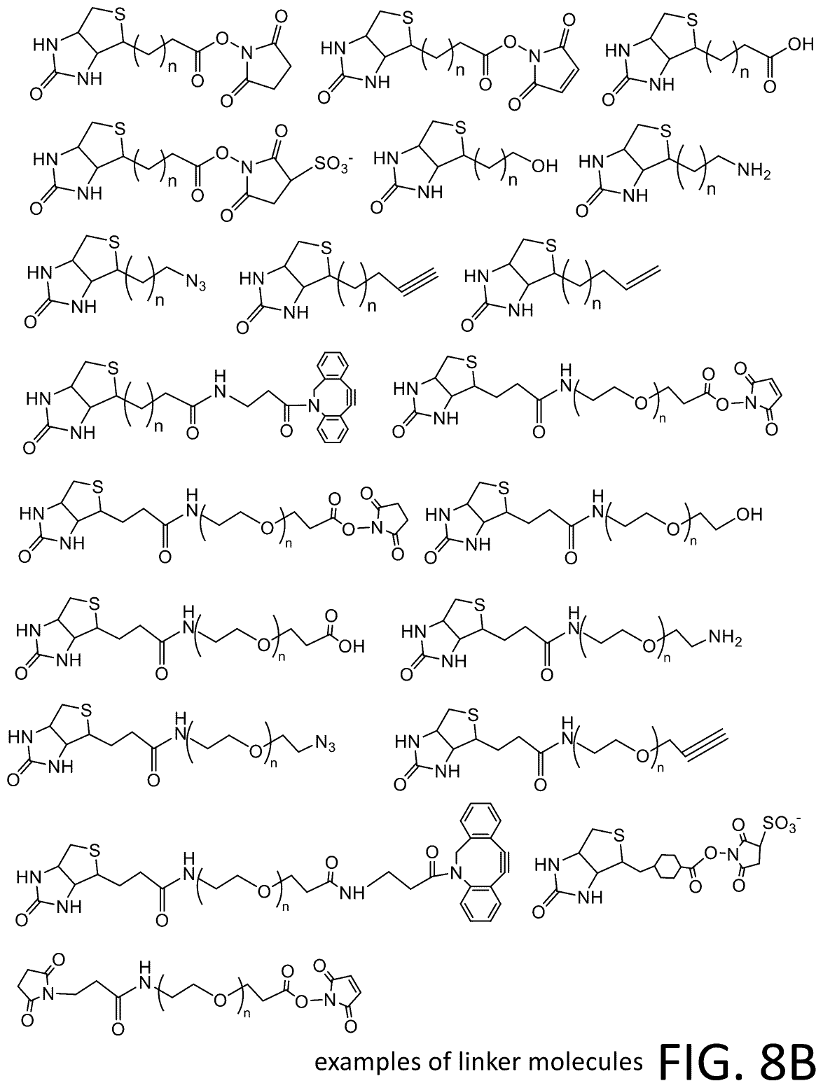

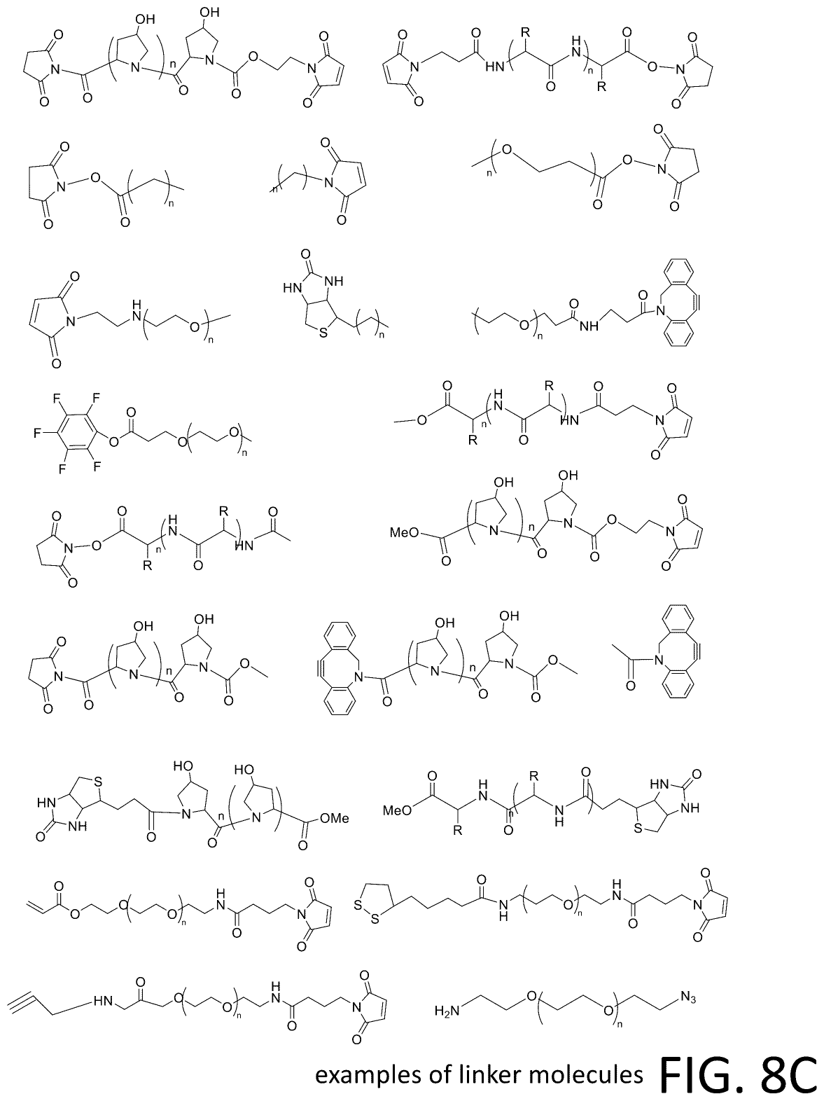

FIGS. 8A-8C show examples of linker molecules. In some embodiments, linker molecules with functional groups include but not limited to mono-functional monomers or oligomers, di-functional monomers or oligomers, tri-functional monomers or oligomers, in some details, above functional monomers or oligomers have functional groups at one end that can react with antibody or other bioactive molecules, the functional group R1 can be thiol, hydroxyl, carboxyl, azide, maleimide, alkyne, biotin, silane, bicyclo[6.1.0]nonyne, dibenzocyclooctyne (DBCO), methyltetrazine, trans-cycloctene (TCO), tetrazine, amine, tetrafluorophenyl esters, succinimidyl ester, pentafluorophenyl ester, bromo, iodol, hydrazide, tosyl, aldehyde, isocyanate, vinyl, epoxide, acrylate, etc. linker molecules also has functional group at the other end that can react with functional groups on surface of inorganic nanoparticle, the functional group R2 can be thiol, hydroxyl, carboxyl, azide, maleimide, alkyne, biotin, bicyclo[6.1.0]nonyne, dibenzocyclooctyne (DBCO), methyltetrazine, trans-cycloctene (TCO), tetrazine, silane, amine, tetrafluorophenyl esters, succinimidyl ester, pentafluorophenyl ester, bromo, iodol, hydrazide, tosyl, aldehyde, isocyanate, vinyl, epoxide, acrylate, etc. Functional groups at each side can be different, can also be same.

In some embodiments, mono-functional monomers or oligomers, di-functional monomers or oligomers, tri-functional monomers or oligomers can be used for the reactions respectively, sometimes, they can also be used at the same time.

A number of examples of nanoparticle fluorescent dye complexes are disclosed below, including nanoparticle-cFluor V420 complexes (SN-cFluor V420), nanoparticle-cFluor UV388 complexes (SN-cFluor UV388), nanoparticle-cFluor B532 complexes (SN-cFluor B532), and nanoparticle-cFluor R780 complexes (SN-cFluor R780). The disclosed nanoparticle fluorescent dye complexes with silane coupling agents were compared with their commercial fluorescent dye counterparts without the silane coupling agent at the same labeling concentration and under identical experimental conditions to provide comparison of brightness, cell labeling efficiency, and overall performance.

### Example—Nanoparticle-cFluor V420 Complexes (SN-cFluor V420)

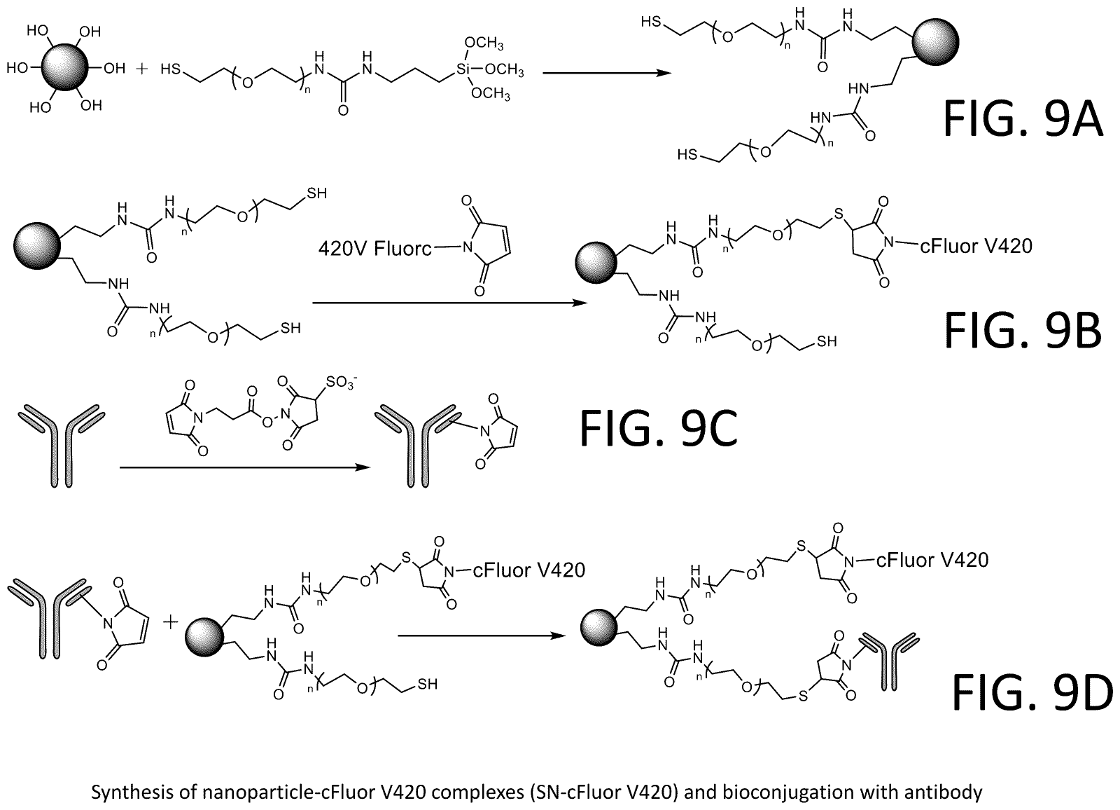

FIGS. 9A-9D illustrate the synthesis of nanoparticle-cFluor V420 complexes (SN-cFluor V420) and bioconjugation with an antibody. The general method of the synthesis of various embodiments of the invention are detailed below corresponding to their illustrations in FIGS. 9A-9D.

### Preparation of Mercapto-Functionalized Silica Nanoparticles

The silane coupling agent, silane-PEG12-SH, was used for the modification of silica nanoparticles. An amount of 1 gram (g) of silica nanoparticles was dispersed in 30 milliliters (ml) of ethanol, and into which 3 mL of water, 2 mL of ammonia (25 wt. %), and 0.6 g of silane-PEG12-SH were added. The mixture was under ultrasonication for 1 hour (hr) and was then stirred at 1000 rotations per minute (rpm) at room temperature for 48 hours. The dispersion was purified from free silane-PEG12-SH and water or ammonia by five cycles of centrifuge and redispersion of the precipitate in absolute ethanol, to finally obtain SH-PEG groups covered silica nanoparticles.

Preparation of cFluor V420 Dye Grafted Silica Nanoparticles (SN-cFluor V420)

Referring to FIG. 9B, 30 mg of mercapto-Functionalized silica nanoparticles are dispersed in 10 mL of PBS buffer. Then, 0.2 mg of maleimide-cFluor V420 dye in DMF is added into the solution and reacted at room temperature for 5 hours to form the cFluor V420 dye grafted silica nanoparticles (SN-cFluor V420).

### Bioconjugation Between SN-cFluor V420 and Activated Antibody

Referring to FIG. 9C, CD4 antibody is activated with NHS-3-maleimidopropionate, forming an exemplary activated antibody of possible activated antibodies. Referring to FIG. 9D, the activated CD4 antibody is then reacted with SN-cFluor V420 at room temperature for 5 hours to form the bioconjugation.

### Full Spectrum Flow Cytometry Tests

Bioconjugation was successfully performed using the silica dyes via click chemistry. CD4-SN-cFluor V420 bioconjugates were used to label whole blood cells. Click chemistry is a method for attaching a probe or substrate of interest to a specific biomolecule, a process called bioconjugation. Click chemistry reactions rely on highly energetic reagents or reactants that are often described as being “spring-loaded”. Click chemistry reactions are the generally modular, wide in scope, give very high yields, generate only inoffensive byproducts that can be removed by non-chromatographic methods, and are stereo-specific (but not necessarily enantioselective.

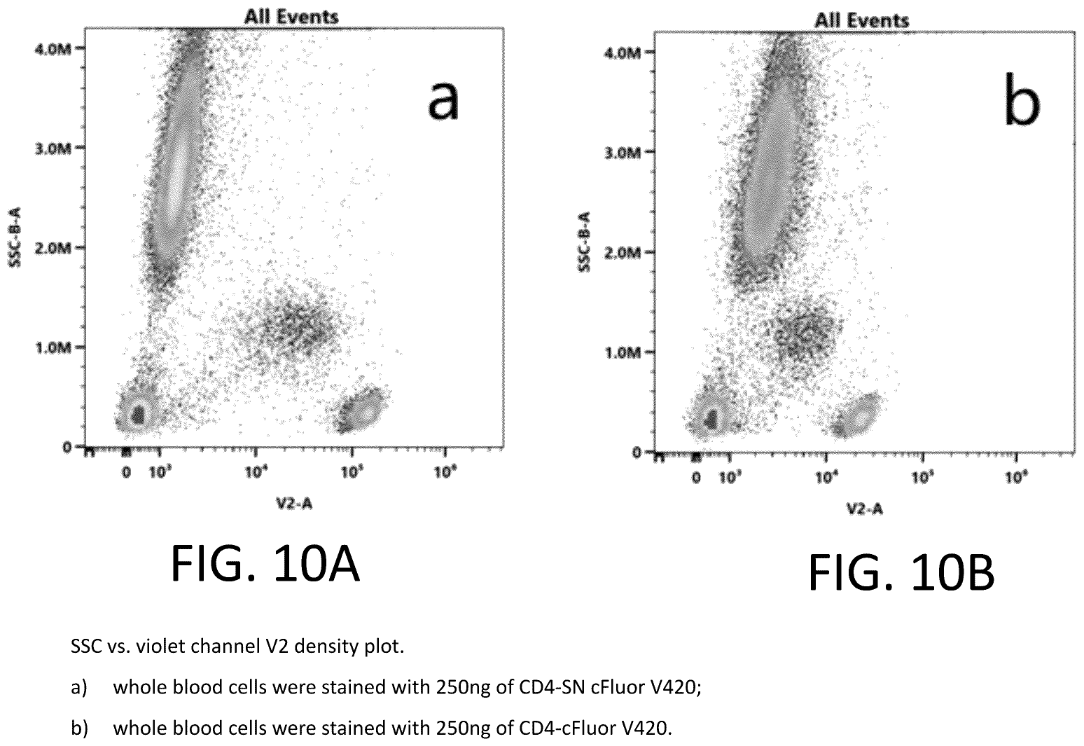

FIGS. 10A-10B, 11A-11B, and 12 show full spectrum flow cytometry test results of CD4-SN-cFluor V420 marker-dye conjugate and CD4-cFluor V420 marker-dye conjugate for comparison. The disclosed nanoparticle fluorescent dye complexes with silane coupling agents were compared with their commercial fluorescent dye counterparts without the silane coupling agent at the same labeling concentration and under identical experimental conditions to provide comparison of brightness, cell labeling efficiency, and overall performance.

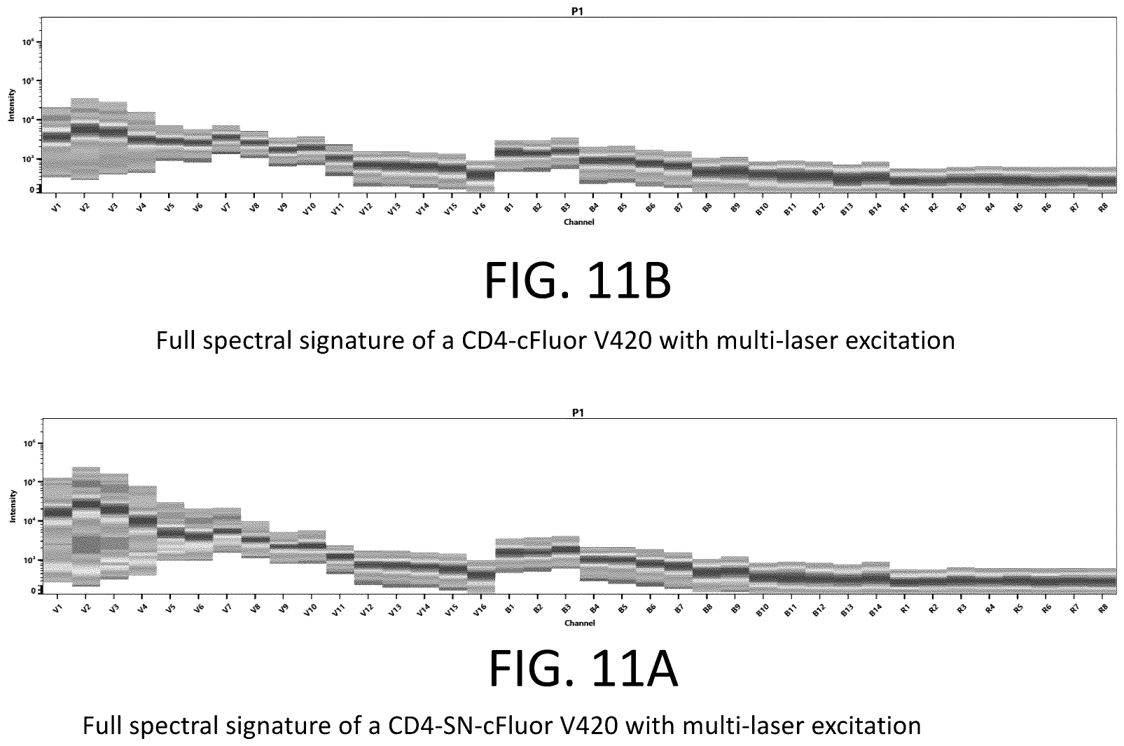

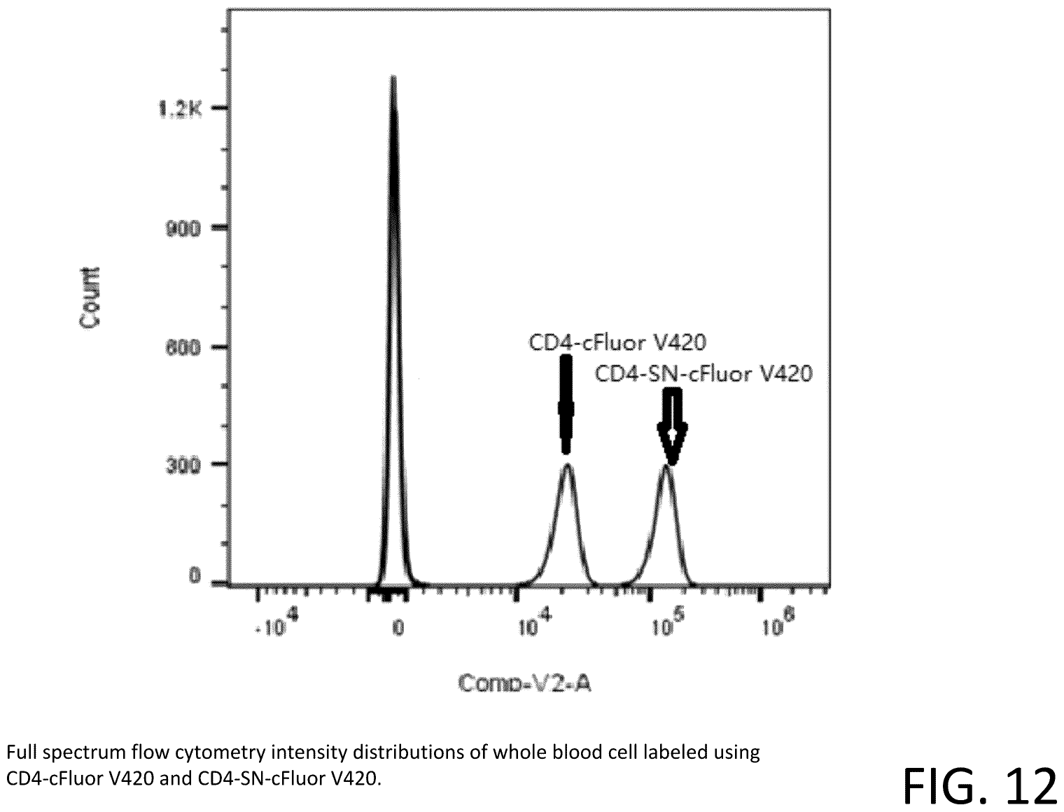

FIG. 10A illustrate a density plot of the side scanner channel (SSC) versus the violet channel V2 for whole blood cells stained with CD4-SN-cFluor V420. FIG. 10B illustrate a density plot of the side scanner channel (SSC) versus the violet channel V2 for whole blood cells stained with CD4-cFluor V420. FIG. 11A illustrates a full spectral signature of CD4-SN-cFluor V420 with multi-laser excitation. FIG. 11B illustrates a full spectral signature of CD4-cFluor V420 with multi-laser excitation. FIG. 12 illustrates plots of full spectrum flow cytometry intensity distributions of whole blood cell labeled using CD4-cFluor V420 and CD4-SN-cFluor V420.

These test results show that CD4-SN-cFluor V420 were effectively labeled on the cell surface and gave a brighter intensity signal (peak amplitude of fluorescence intensity) than CD4-cFluor V420.

FIG. 12 for example, shows that for the same count (Y-axis), CD4-SN-cFluor V420 marker-dye conjugate is about six times brighter (X-axis) than the commercial CD4-CFluor V420 marker-dye conjugate.

### Examples Nanoparticle-cFluor UV388 Complexes (SN-cFluor UV388)

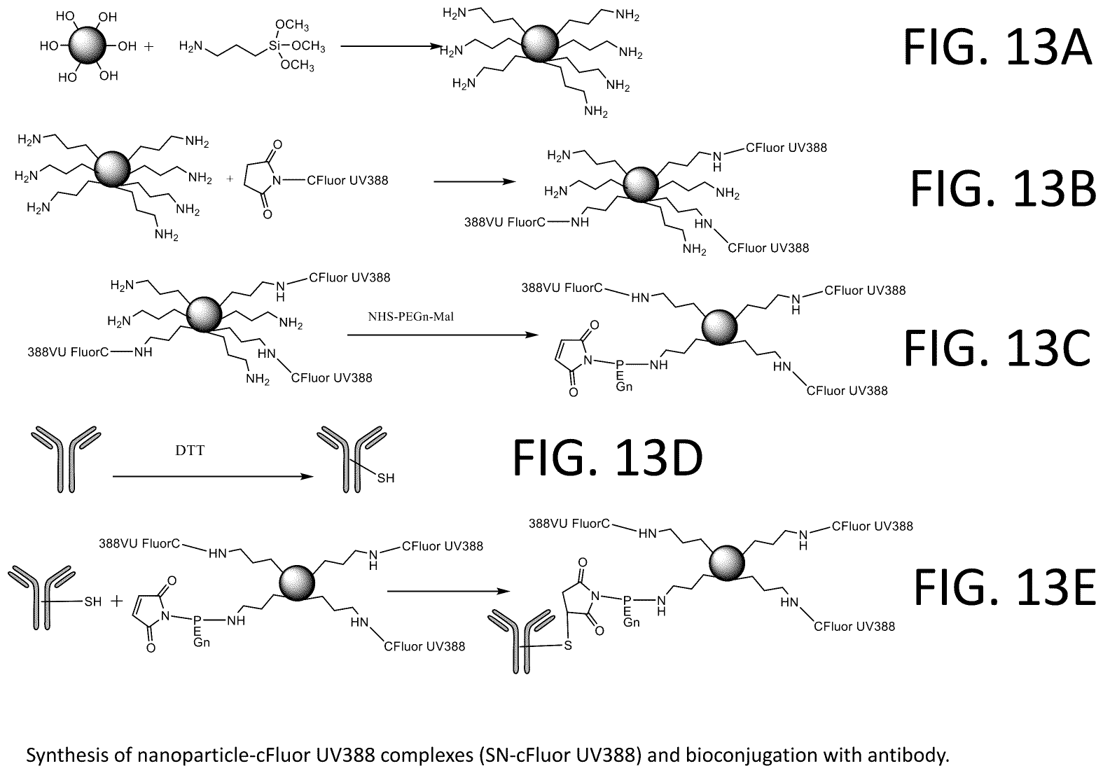

FIGS. 13A-13E illustrate synthesis of nanoparticle-cFluor UV388 complexes (SN-cFluor UV388) and bioconjugation with an antibody.

### Preparation of Amino-Functionalized Silica Nanoparticles

Referring to FIG. 13A, A silane coupling agent (SN), 3-aminopropyl trimethoxysilane (APTMS), is used for the modification of silica nanoparticles. A mixture containing 2.5 mmol of APTMS, 0.1 mL of 28 percentage weight (wt. %) ammonia and 1.5 mL of deionized water is slowly added to the silica nanoparticles suspensions under vigorous stirring. The amination reaction is carried out on the surface of the silica nanoparticles. The final concentration of each species is 0.03 molar (M) for APTMS, 0.02 M for NH₃, 0.8 M for H₂O, and 20 M for ethanol, respectively. In order to enhance the covalent bonding between APTMS and silica surface, the resultant solution is stirred and refluxed at 75° C. for 8 hours to obtain a white-blue suspension. The visible white precipitates are the centrifuged and washed with ethanol and water 6 times followed by vacuum drying at 50° C. for 12 hours to obtain the aminopropyl Functionalized silica nanoparticles.

Preparation of cFluor UV388 Dye Grafted Silica Nanoparticles (SN-cFluor UV388)

Referring now to FIG. 13B, 20 mg of aminopropyl-Functionalized silica nanoparticles are dispersed in 4 mL of ethanol solution. An NHS-cFluor UV388 dye in DMSO is then added into the solution and react at room temperature for 2 hours to form the cFluor UV388 dye grafted silica nanoparticles (SN-cFluor UV388).

### Functionalization of SN-cFluor UV388

Referring now to FIG. 13C, 0.1 mg of NHS-PEG-Malemide in DMSO is added into above solution containing cFluor UV388 dye grafted silica nanoparticles (SN-cFluor UV388) and reacted at room temperature for 2 hours. 1 mg of N-Acetoxysuccinimide in DMSO is then added into the solution and reacted for an additional 2 hours. The final solution is then centrifuged and the functionalized SN-cFluor UV388 is then dissolved in a PBS buffer.

### Bioconjugation Between SN-cFluor UV388 and Activated Antibody

Referring now to FIG. 13D, a CD4 antibody is activated with DL-Dithiothreitol (DTT). Referring to FIG. 13E, the activated CD4 antibody is then reacted with the functionalized SN-cFluor UV388 at room temperature for 2 hours to bioconjugate them together.

### Full Spectrum Flow Cytometry Test

Bioconjugation was successfully performed using the silica dyes via click chemistry. CD4-SN-cFluor UV388 bioconjugates were used to label whole blood cells.

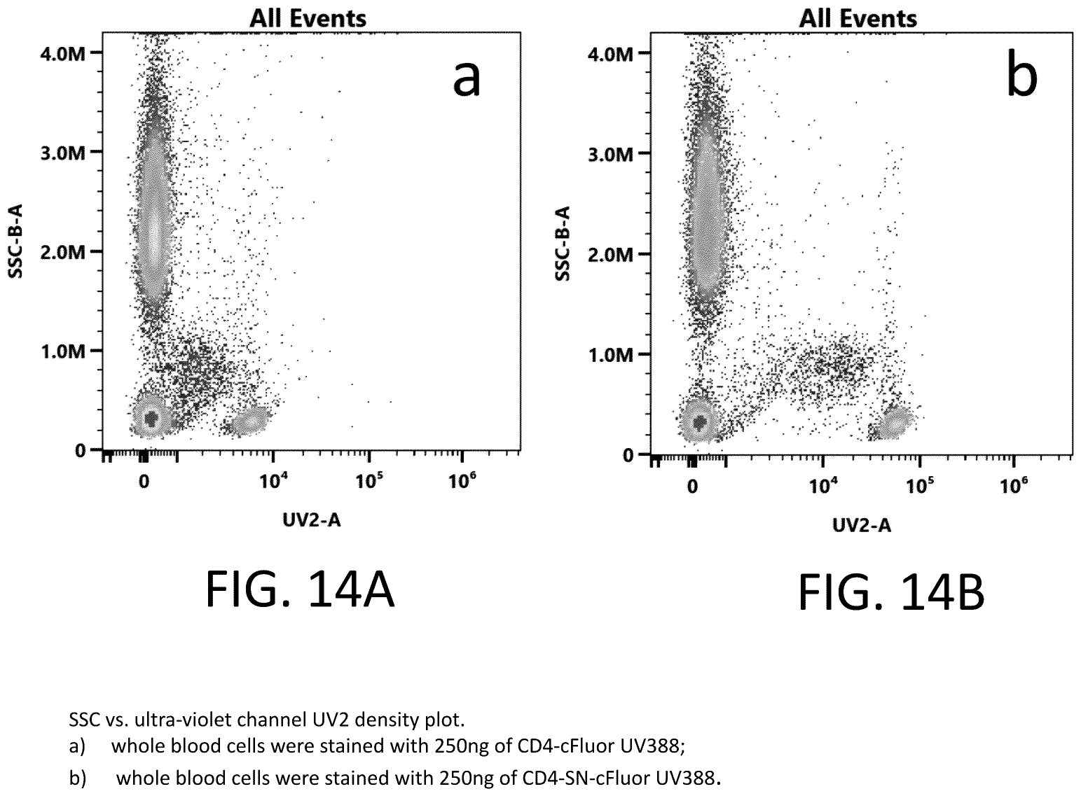

FIGS. 14A-14B, 15A-15B, and 16 show full spectrum flow cytometry test results of CD4-SN-cFluor UV388 marker-dye conjugate and CD4-cFluor UV388 marker-dye conjugate for comparison.

FIG. 14A illustrates a density plot of the side scanner channel (SSC) versus ultra-violet channel UV2 for whole blood cells stained with CD4-cFluorUV388. FIG. 14B illustrates a density plot of the side scanner channel (SSC) vs. ultra-violet channel UV2 for whole blood cells stained with CD4-SN-cFluorUV388.

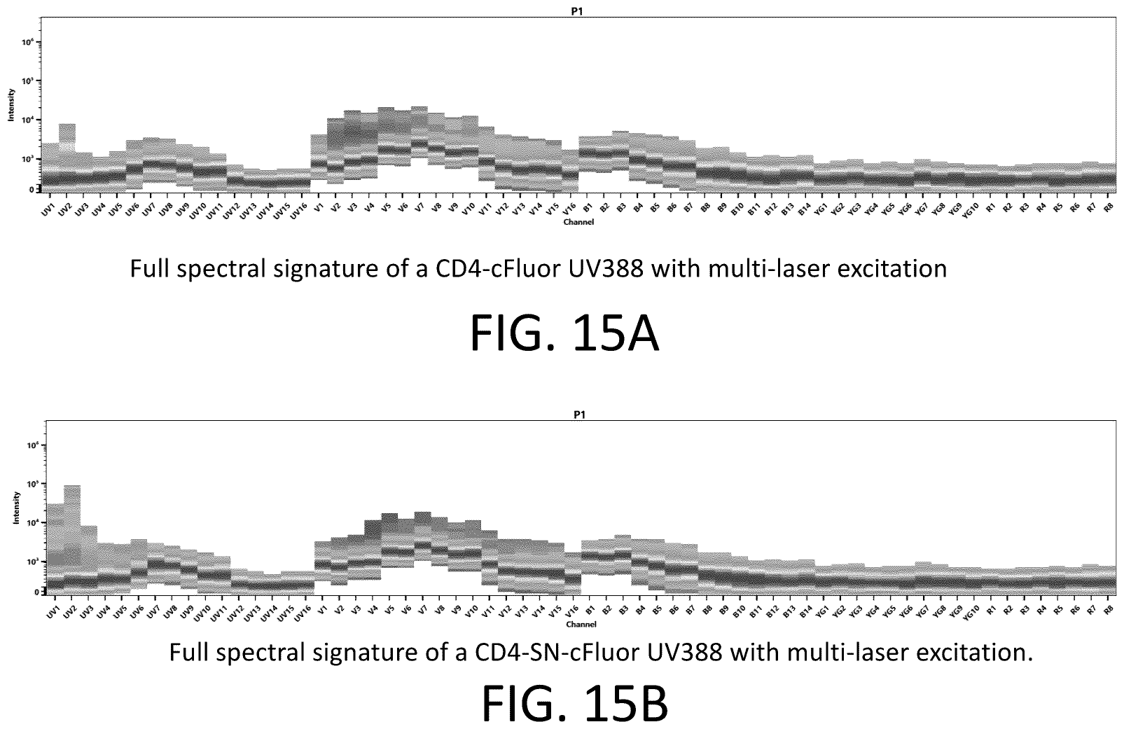

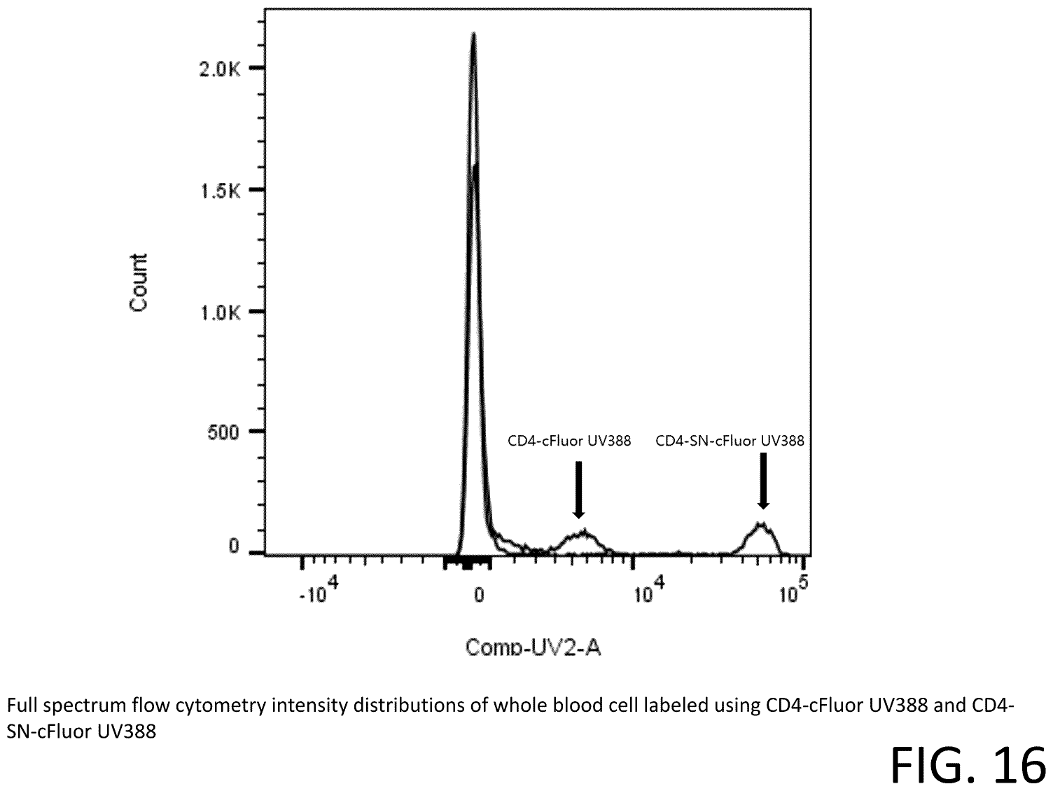

FIG. 15A illustrates a full spectral signature of a CD4-cFluor UV388 with multi-laser excitation. FIG. 15B illustrates a full spectral signature of a CD4-SN-cFluor UV388 with multi-laser excitation. FIG. 16 illustrates plots of full spectrum flow cytometry intensity distributions of whole blood cells labeled using CD4-cFluor UV388 and CD4-SN-cFluor UV388.

The full spectrum flow cytometry results shown in FIGS. 14A-14B, 15A-15B, and 16 indicate that CD4-SN-cFluor UV388 probes were effectively labeled on the cell surface and gave brighter fluorescence signal. The test results of SN-cFluor UV388 dye with the commercial cFluor UV388 dye were at the same labeling concentration and under identical experimental conditions. Accordingly, the test results provide a comparison of the brightness, cell labeling efficiency, and overall performance between SN-cFluor UV388 dye with the commercial cFluor UV388 dye.

FIG. 16 for example, shows that for the about the same count (Y-axis), CD4-SN-cFluor UV388 is about nine times brighter (intensity along X-axis) than commercial CD4-cFluor UV388.

### Example—Nanoparticle-cFluor B532 Complexes (SN-cFluor B532)

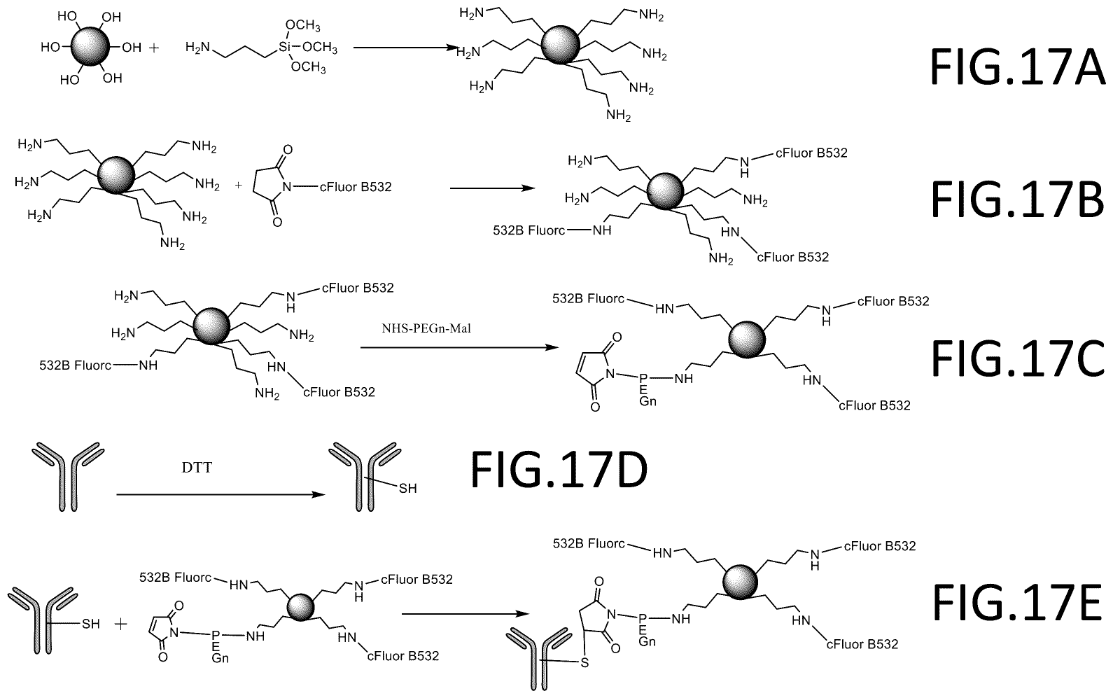

FIGS. 17A-17E illustrate synthesis of nanoparticle-cFluor B532 complexes (SN-cFluor B532) and bioconjugation with antibody.

Preparation of cFluor B532 Dye Grafted Silica Nanoparticles (SN-cFluor B532)

In FIG. 17A, 30 mg of amino-Functionalized silica nanoparticles are dispersed in 3 mL of an ethanol solution. In FIG. 17B, NHS-cFluor B532 dye in DMSO is then added into solution and reacted at room temperature for 2 hours to get the cFluor B532 dye grafted silica nanoparticles (SN-cFluor B532).

### Functionalization of SN-cFluor B532

In FIG. 17C, 0.2 mg of NHS-PEG-Malemide in DMSO is added into the above solution containing SN-cFluor B532 and reacted at room temperature for 2 hours. Then 1 mg of N-Acetoxysuccinimide in DMSO is added into the solution and reacted for additional 2 hours. The final solution is then centrifuged and the functionalized SN-cFluor B532 dye is dissolved in a PBS buffer.

### Bioconjugation Between SN-cFluor B532 and Activated Antibody

In FIG. 17D, a CD4 antibody is activated with DL-Dithiothreitol (DTT). In FIG. 17E, the activated CD4 antibody is then reacted with SN-cFluor B532 at room temperature for 1 hour to form the bioconjugation.

### Full Spectrum Flow Cytometry Test

Bioconjugation was successfully performed using the silica dyes via click chemistry. CD4-SN-CFluor B532 bioconjugates were used to label whole blood cells.

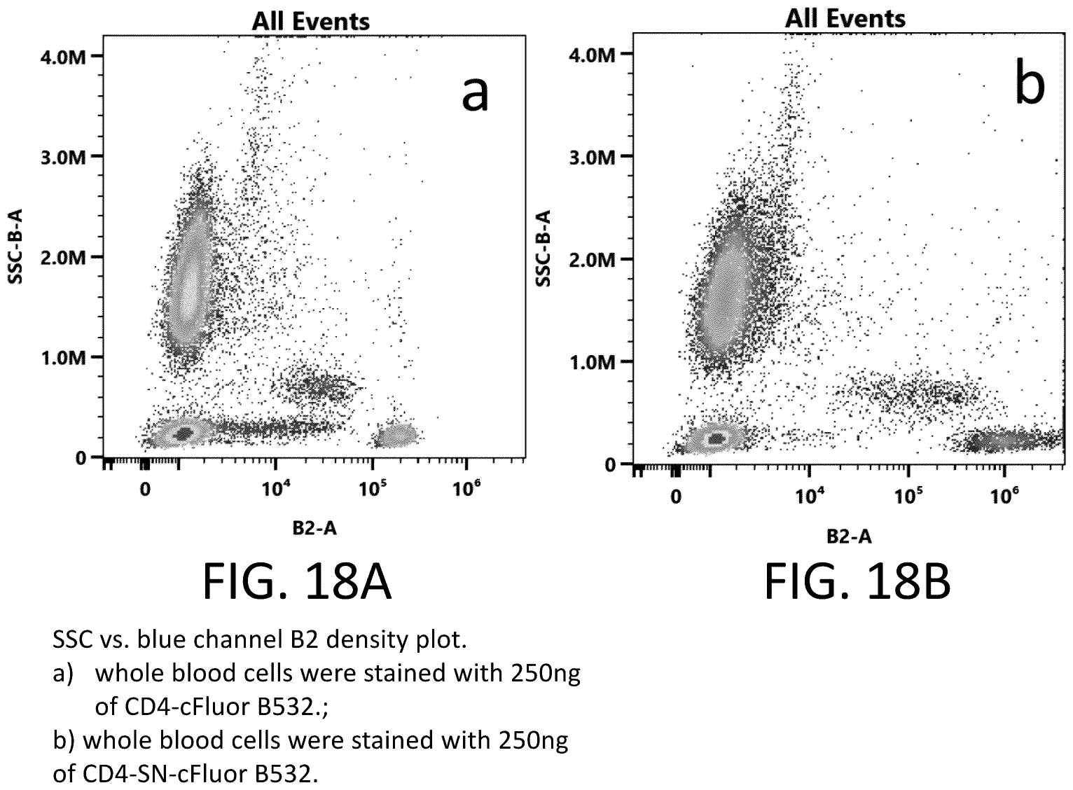

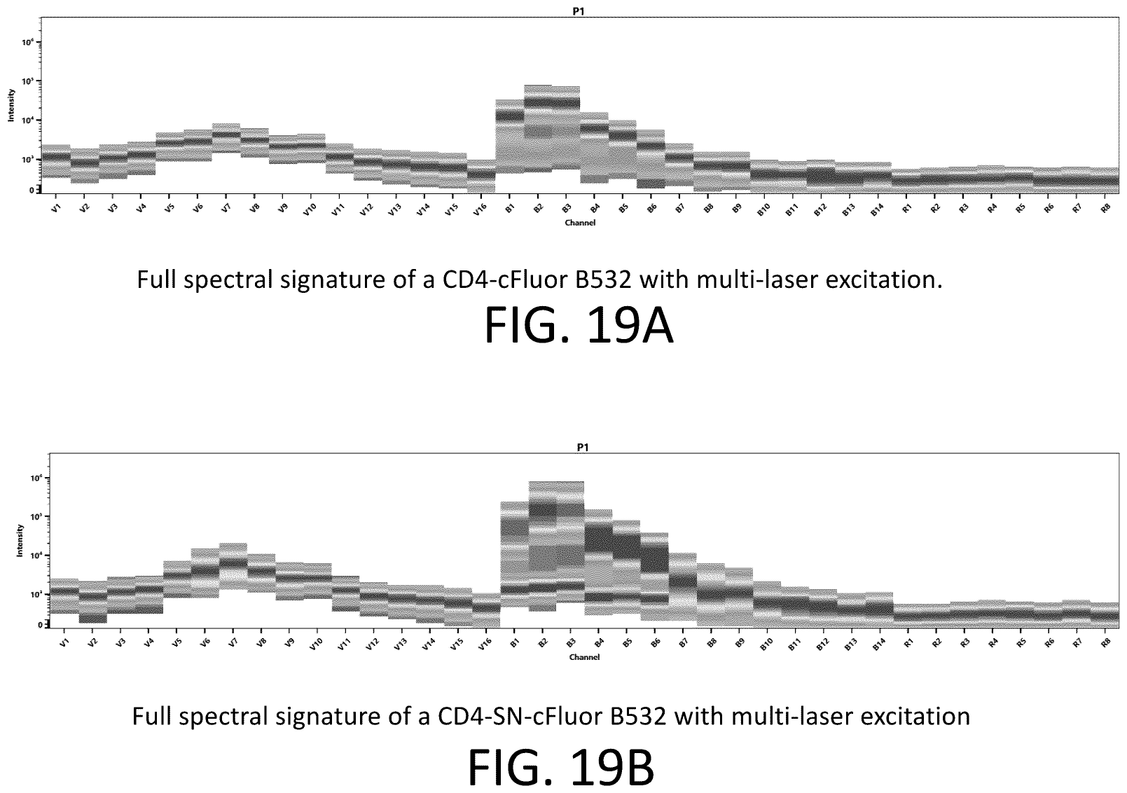

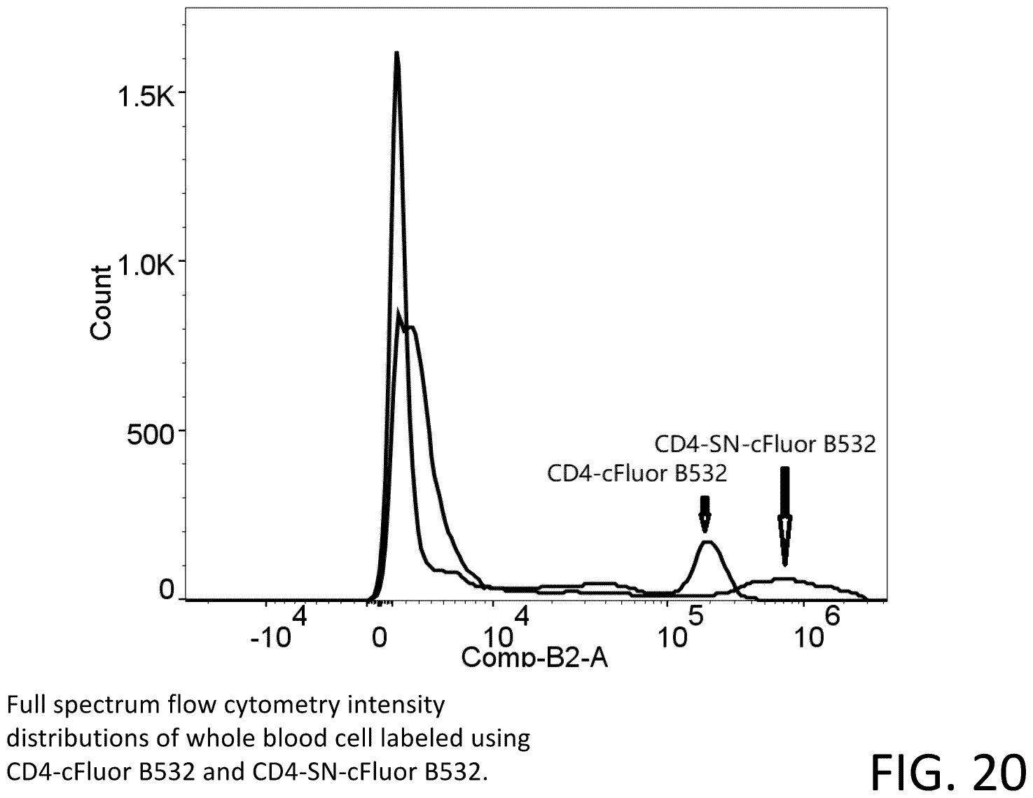

FIGS. 18A-18B, 19A-19B, and 20 show full spectrum flow cytometry test results of CD4-SN-cFluor B532 marker-dye conjugate and CD4-cFluor B532 marker-dye conjugate for comparison. FIG. 18A illustrates a density plot of the side scanner channel (SSC) versus blue channel B2 for whole blood cells stained with CD4-cFluor B532. FIG. 18B. illustrates a density plot of the side scanner channel (SSC) versus blue channel B2 for whole blood cells stained with CD4-SN-cFluor B532. FIG. 19A illustrates a full spectral signature of a CD4-cFluor B532 with multi-laser excitation. FIG. 19B illustrates a full spectral signature of a CD4-SN-cFluor B532 with multi-laser excitation. FIG. 20 illustrates plots of full spectrum flow cytometry intensity distributions of whole blood cell labeled using CD4-cFluor B532 and CD4-SN-cFluor B532.

FIGS. 18A-18B, 19A-19B, and 20 indicate that CD4-SN-cFluor B532 probes (dye) with the silane linkage is effectively labeled on the cell surface and gave a brighter fluorescence signal than that of the commercial cFluor B532 dye without silane linkage. These comparisons were made with the same labeling concentration and under identical experimental conditions to provide a further comparison of their brightness, cell labeling efficiency, and overall performance.

FIG. 20 indicates that CD4-SN-cFluor B532 with silane linkage, even having lower counts (Y-axis count), is about six times brighter (X-axis-intensity) than the commercial CD4-cFluor B532 dye without silane linkage.

### Example—Nanoparticle-cFluor R780 Complexes (SN-cFluor R780)

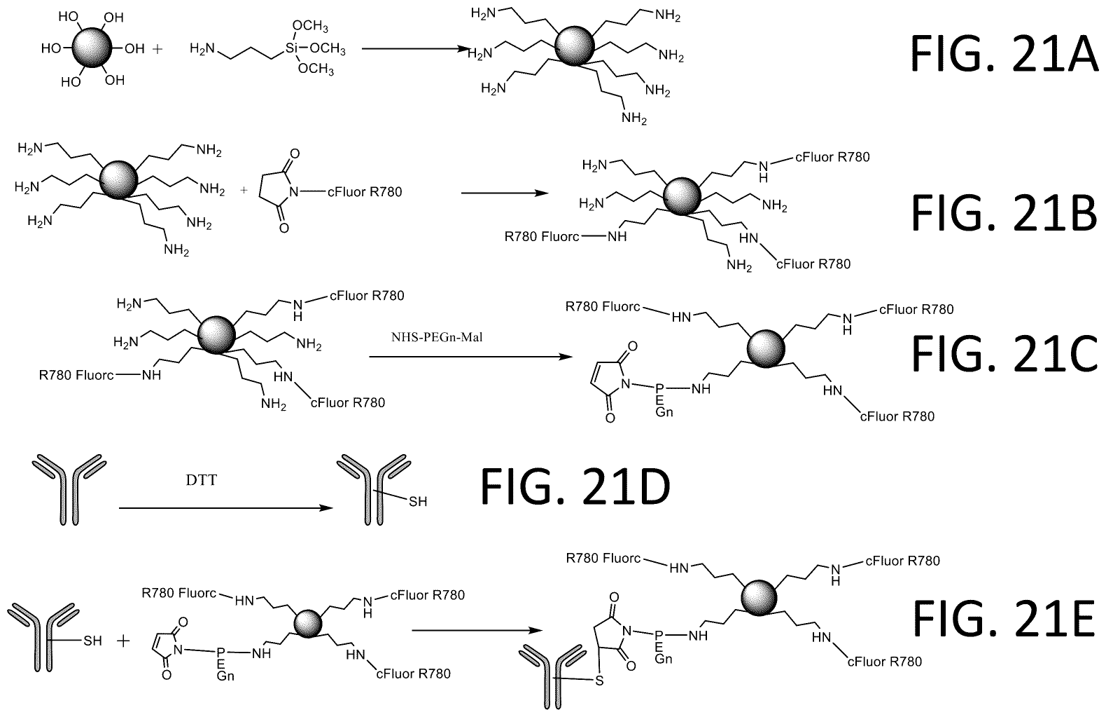

FIGS. 21A-21E illustrate synthesis of nanoparticle-cFluor R780 complexes (SN-cFluor B780) and bioconjugation with an antibody.

Preparation of cFluor R780 dye grafted silica nanoparticles (SN-cFluor R780) is shown in FIGS. 21A-21B.

In FIG. 21A, 20 milligrams (mg) of amino-Functionalized silica nanoparticles are dispersed into 3 mL of an ethanol solution. As shown in FIG. 21B, NHS-cFluor R780 dye in DMSO is then added into the solution and reacted at room temperature for 2 hours to form the cFluor R780 dye grafted silica nanoparticles (SN-cFluor R780).

### Functionalization of SN-cFluor R780

In FIG. 21C, 0.2 mg of NHS-PEG-Malemide in DMSO is added into the solution of the cFluor R780 dye grafted silica nanoparticles (SN-cFluor R780) and reacted at room temperature for 2 hours. Then 1 mg of N-Acetoxysuccinimide in DMSO is added in solution and reacted for additional 2 hours. The final solution is then centrifuged and the functionalized SN-cFluor R780 dye is dissolved in a PBS buffer.

### Bioconjugation Between SN-cFluor R780 and Activated Antibody

In FIG. 21D, a CD4 antibody is activated with DL-Dithiothreitol (DTT). In FIG. 21E, the CD4 antibody is then reacted with the functionalized SN-cFluor R780 dye at room temperature for 1 hour.

### Full Spectrum Flow Cytometry Test

Bioconjugation was successfully performed using the silica dyes via click chemistry. CD4-SN-cFluor R780 bioconjugates were used to label whole blood cells.

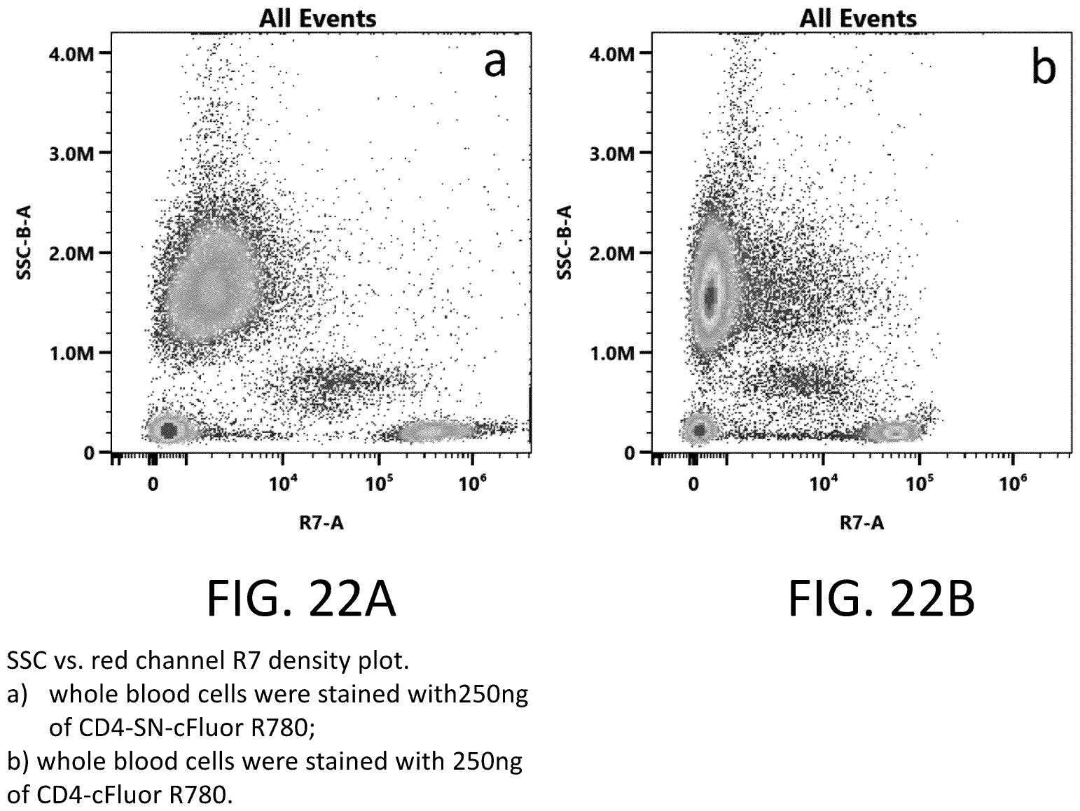

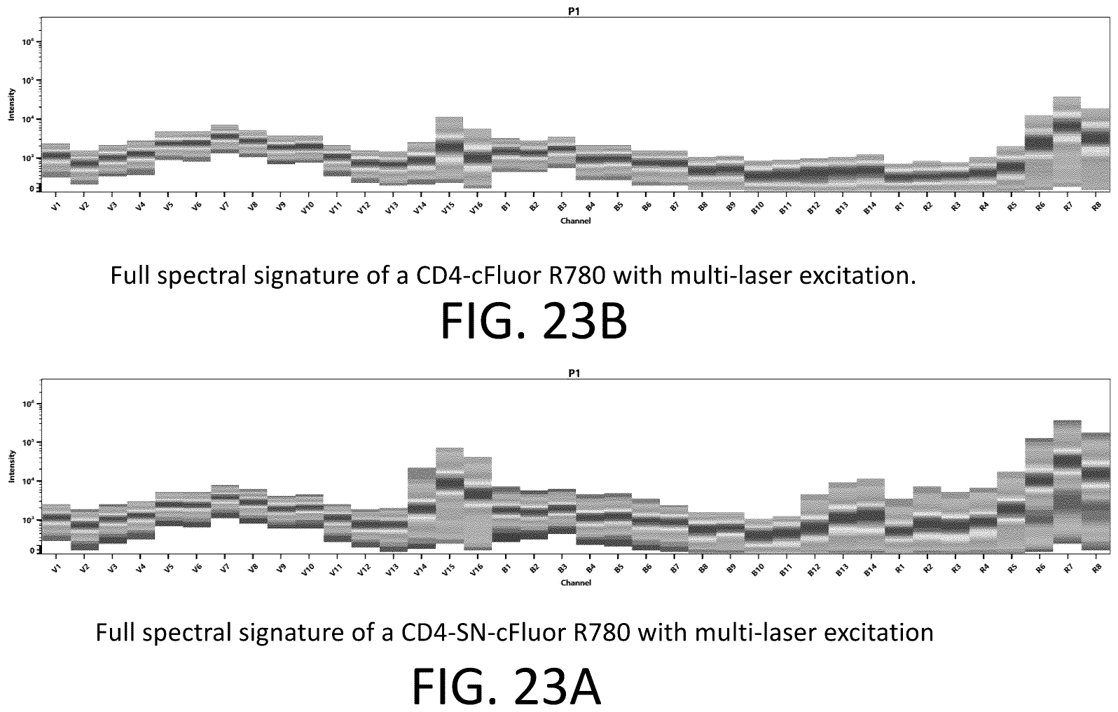

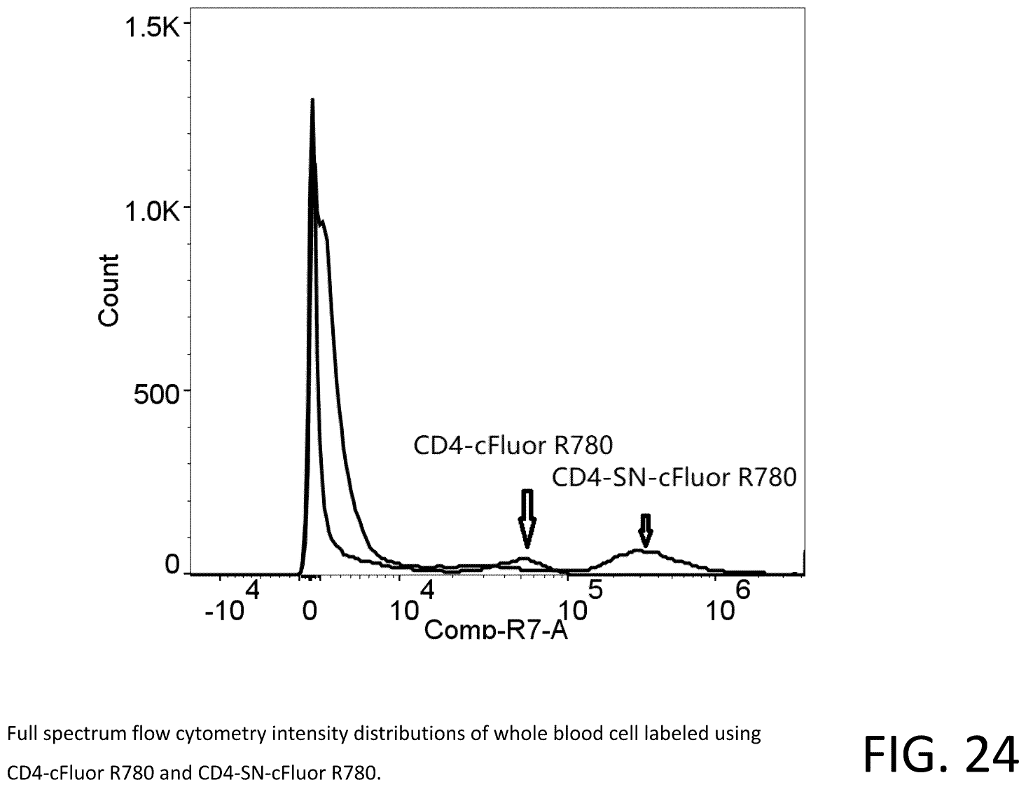

FIGS. 22A-22B, 23A-23B, and 24 show full spectrum flow cytometry test results of CD4-SN-cFluor R780 marker-dye conjugate and CD4-cFluor R780 marker-dye conjugate for comparison. FIG. 22A illustrates a density plot of the side scanner channel (SSC) versus red channel R7 density plot for whole blood cells stained with CD4-SN-cFluor R780. FIG. 23B illustrates a density plot of the side scanner channel (SSC) versus red channel R7 for whole blood cells stained with CD4-cFluor R780. FIG. 23A illustrates a plot of a full spectral signature of a CD4-SN-cFluor R780 with multi-laser excitation. FIG. 22B illustrates a plot of a full spectral signature of a CD4-cFluor R780 with multi-laser excitation. FIG. 24 illustrates plots of full spectrum flow cytometry intensity distributions of whole blood cell labeled using CD4-cFluor R780 and CD4-SN-cFluor R780.

FIGS. 22A-22B, 23A-23B, and 24 show the full spectrum flow cytometry results, which proved that CD4-SN-cFluor R780 were effectively labeled on the cell surface and gave brighter fluorescence signal. We compared our SN-cFluor R780 with cFluor R780 dye at the same labeling concentration and under identical experimental conditions to provide a further comparison of their brightness, cell labeling efficiency, and overall performance.

FIG. 24 for example, shows that CD4-SN-cFluor R780 is about six times brighter (X-axis intensity) than CD4-cFluor R780.

### Reagent Kit

A single-tube reagent kit disclosed herein can demonstrate an effective, high sensitivity flow cytometry approach that can be used for monitoring immune cell subsets in blood in clinical and translational research.

The biological samples to which this reagent kit is used can be peripheral blood samples and bone marrow samples. The reagent composition of a conjugated inorganic nanoparticle fluorescent dye complexes and antibodies are shipped in a plurality (e.g., one vial per color-20 or more for other included chemicals) of sealable test tubes (vials) in a box with instructions of use, they can be mixed together in one sample test tube with a peripheral blood sample or a bone marrow sample for running through the flow cytometer to quickly obtain results of cell counts and further information (e.g., size, shape, etc.) about the cells in the sample.

A reagent kit embodiment of the invention containing a plurality of reference reagents (2 μl/test) for multiple tests per vial comprising conjugated inorganic nanoparticle fluorescent dye complexes and clones can be utilized to identify subpopulation differential experiments. Embodiments of the reagent kit can contain the aforementioned multiple single reference reagents or a multicolor antibody cocktail combining all multiple single reagents into one sample tube.

### Flow Cytometer

Full spectrum flow cytometry is a technology that enables the development of such highly multiparametric panels. A full spectrum flow cytometer measures the entire fluorochrome emission, from ultra-violet to near infra-red, across multiple lasers using many more detectors compared to a conventional flow cytometer. It produces very specific spectral fingerprints that are used to mathematically distinguish one fluorophore from another, even when their maximum emissions (the primary component measured by a conventional flow cytometer) are very similar. Leveraging this full spectrum technology, the ability to combine thirty or more fluorescently labeled antibodies becomes possible using a fluorescence-based full spectrum flow cytometer.

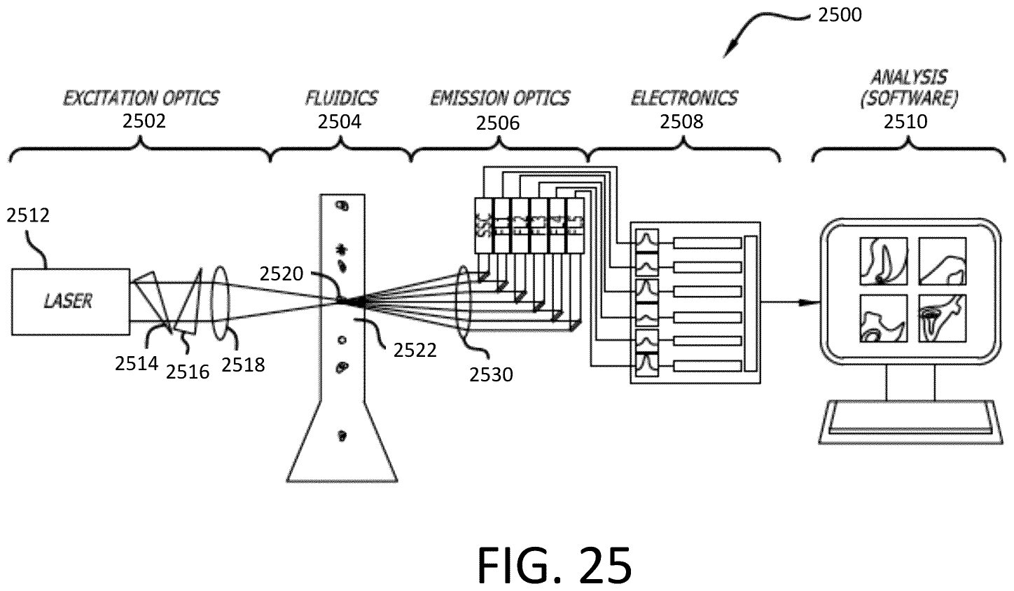

Referring now to FIG. 25, a basic conceptual diagram of a flow cytometer system 2500 is shown. Various embodiments of the flow cytometer 2500 may be commercially available. Five major subsystems of the flow cytometer system 2500 include an excitation optics system 2502, a fluidics system 2504, an emission optics system 2506, an acquisition system 2508, and an analysis system 2510. Generally, a “system” includes hardware devices, software devices, or a combination thereof.

The excitation optics system 2502 includes, for example, a laser device 2512, an optical element 2514, an optical element 2516, and an optical element, 2518. Example optical elements include an optical prism and an optical lens. The excitation optics system 2502 illuminates an optical interrogation region 2520. The fluidics system 2504 carries fluid samples 2522 through the optical interrogation region 2520. The emission optics system 2506 includes, for example, an optical element 2530 and optical detectors SSC, FL1, FL2, FL3, FL4, and FL5. The emission optics system 2506 gathers photons emitted or scattered from passing particles. The emission optics system 2506 focuses these photons onto the optical detectors SSC, FL1, FL2, FL3, FL4, and FL5. Optical detector SSC is a side scatter channel. Optical detectors FL1, FL2, FL3, FL4, and FL5 are fluorescent detectors may include band-pass, or long-pass, filters to detect a particular fluorescence wavelength. Each optical detector converts photons into electrical pulses and sends the electrical pulses to the acquisition system 2508. The acquisition system 2508 processes and prepares these signals for analysis in the analysis system 2510.

The analysis system 2510 can store digital representations of the signals for analysis after completion of acquisition. The analysis system 2510 is a computer with a processor, memory, and one or more storage devices that can store and execute analysis software to obtain laboratory results of biological samples (or other types of samples, e.g., chemical) that are analyzed. The analysis system 2510 can be further used to calibrate the flow cytometer with compensation controls when initialized, before running a reference sample through the flow cytometer. Reference samples can be formed in different ways to determine spillover vectors for a fluorescent dye or fluorochrome. A fluorochrome can be conjugated with an antibody and then attached to a biological cell or attached to a bead or particle.

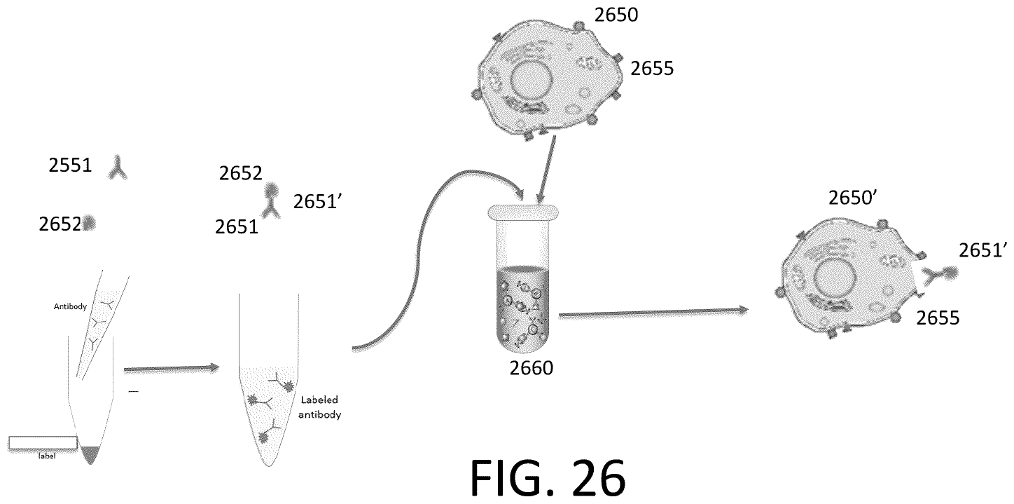

Referring now to FIG. 26, a cell 2650, an antibody 2651, and a fluorochrome (dye) 2652 are coupled together to form a reference sample with direct marking or staining of a cell. The cell 2650 has one or more cell marker 2655 sites to which an antibody can attach. The fluorochrome (dye) 2652 is conjugated with the antibody 2651 in advance to form a conjugated antibody 2651′. For a reference sample, a single fluorochrome (dye) 2652 is conjugated with a single antibody to generate a spillover vector. Subsequently, when analyzing a biological fluid with different unknown counts of cells in the biological fluid, multiple conjugated antibodies with different antibodies and different fluorochrome, can be used and add into the same biological sample.

The conjugated antibodies 2651′ and the cells 2650 are mixed together in a test tube 2660 so the conjugated antibodies 2651′ can attached to the desired cell marker sites 2655 for the given type of cells 2650 to form marked or stained cells 2650′ in the sample biological fluid. When run through the flow cytometer, the fluorochromes can be excited by laser light to fluoresce so that the fluorescence can be detected by detectors as events generating an event vector. The event vector can be used to generate a spill over matrix for the fluorochrome. When running a sample biological fluid with unknown counts, the cells counted by a flow cytometer by analyzing the events.

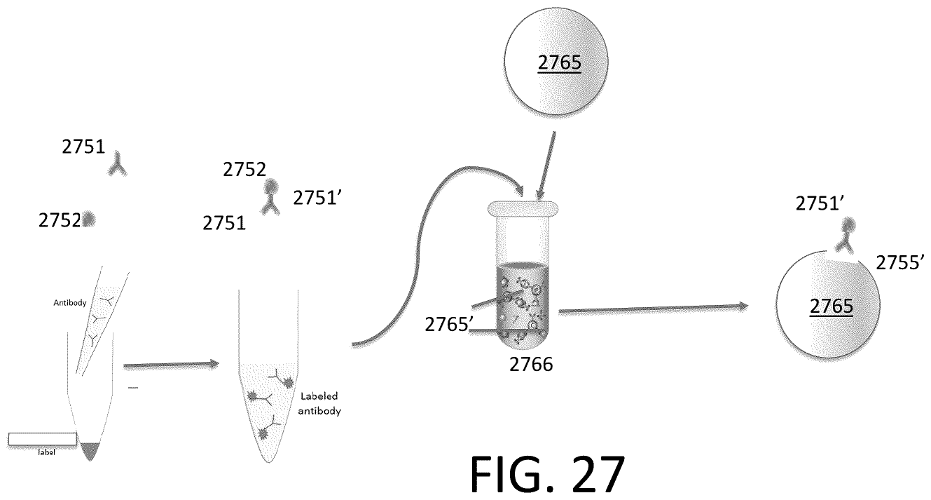

Referring now to FIG. 27, a conceptual diagram of forming a reference sample with a bead 2765 is shown. A bead 2765, an antibody 2751, and a fluorochrome (dye) 2752 are coupled together to form a reference sample with a bead. The bead 2765 may have one or more cell marker 2755′ sites to which an antibody can attach. As with the cell, the fluorochrome (dye) 2752 is conjugated with the antibody 2751 in advance to form a conjugated antibody 2751′. For a reference sample, a single fluorochrome (dye) 2752 is conjugated with a single antibody to generate a spillover vector.

The conjugated antibodies 2751′ and the beads 2765 are mixed together in a test tube 2766 so the conjugated antibodies 2751′ can attached to the desired marker sites 2755′ for the beads 2765 to form marked beads 2765′ in a reference sample. When run through the flow cytometer, the fluorochromes can be excited by laser light to fluoresce so that the fluorescence can be detected by detectors as events generating an event vector. The event vector can be used to generate a spill over matrix for the fluorochrome. In this manner, either cells or beads can be used to test and fluorochrome for suitability to be used with a flow cytometer.

### Reference Sample Run

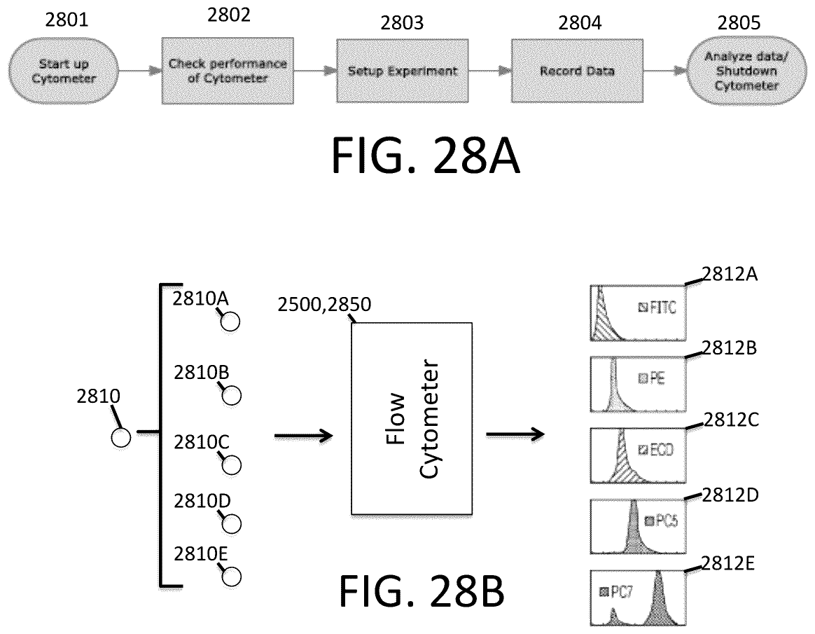

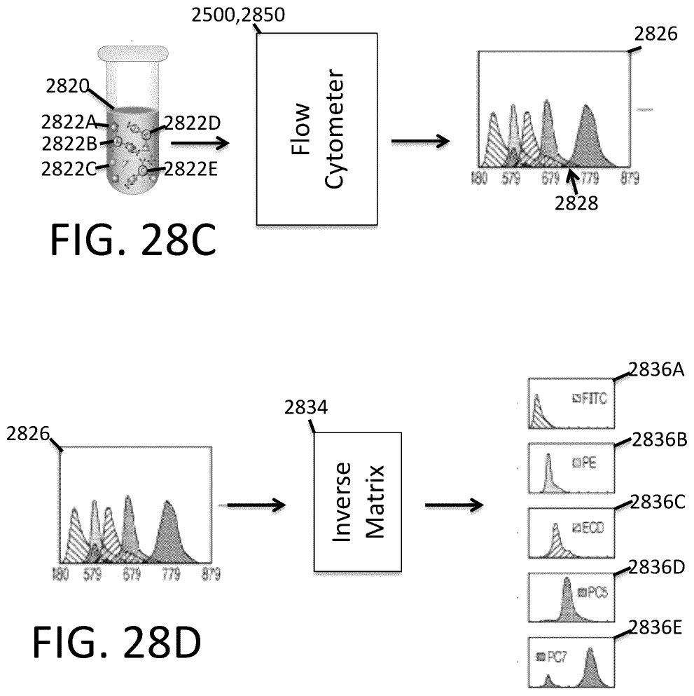

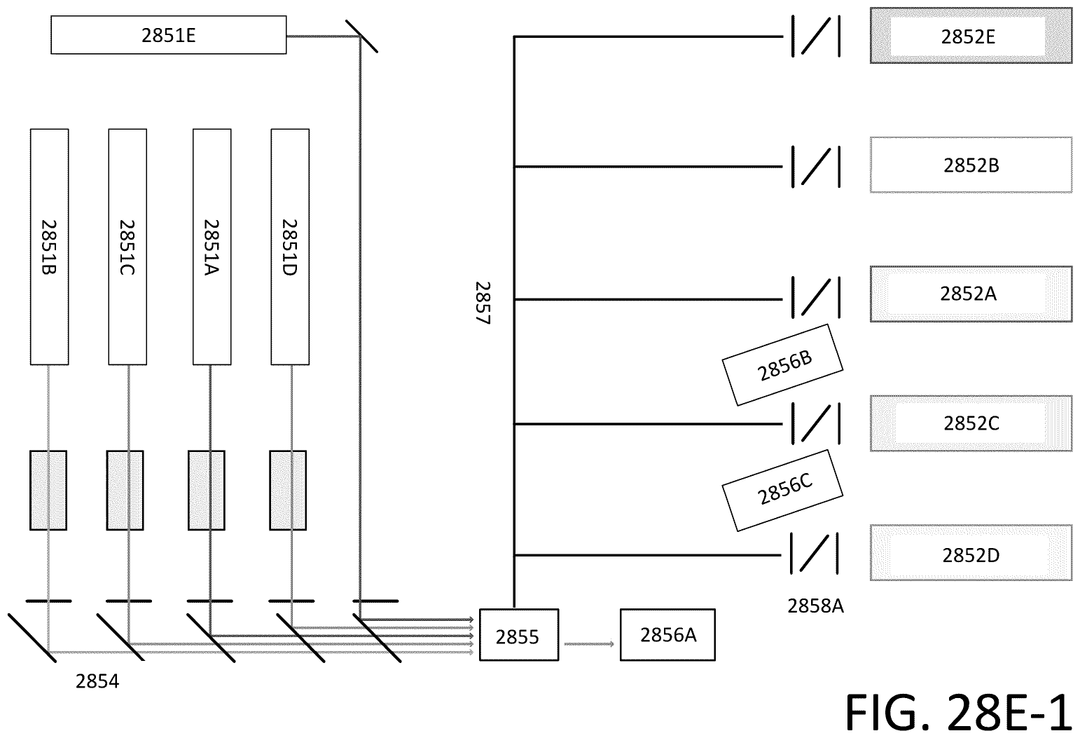

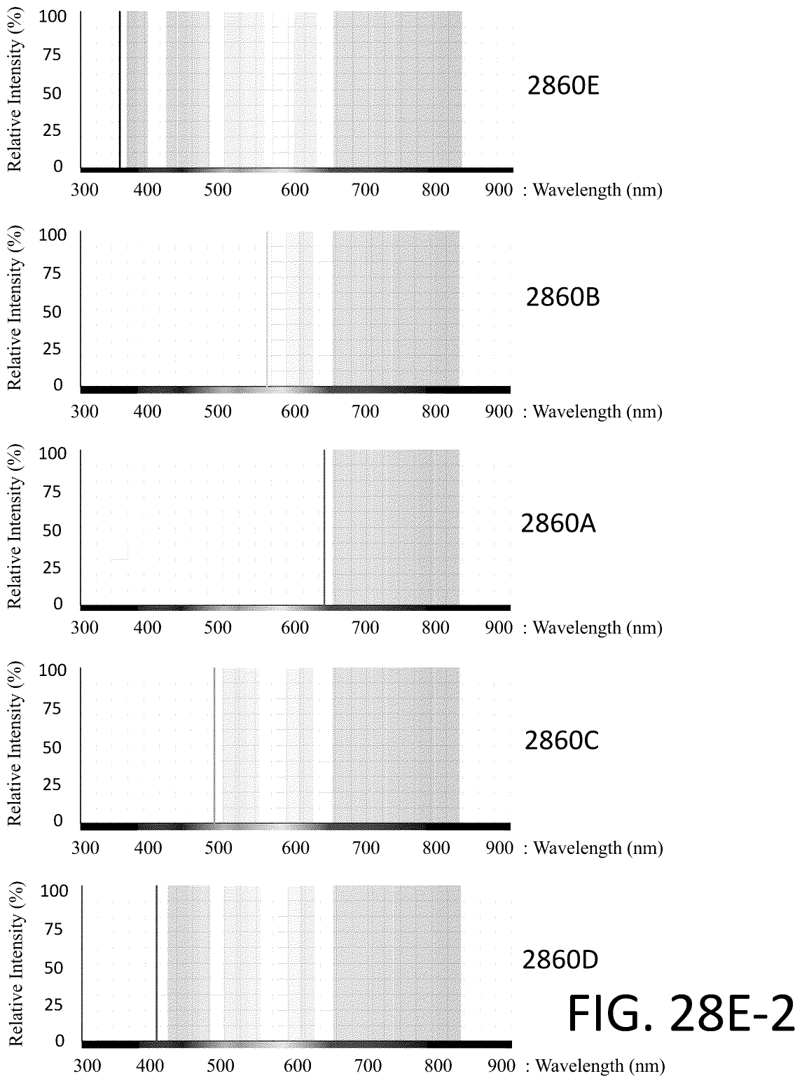

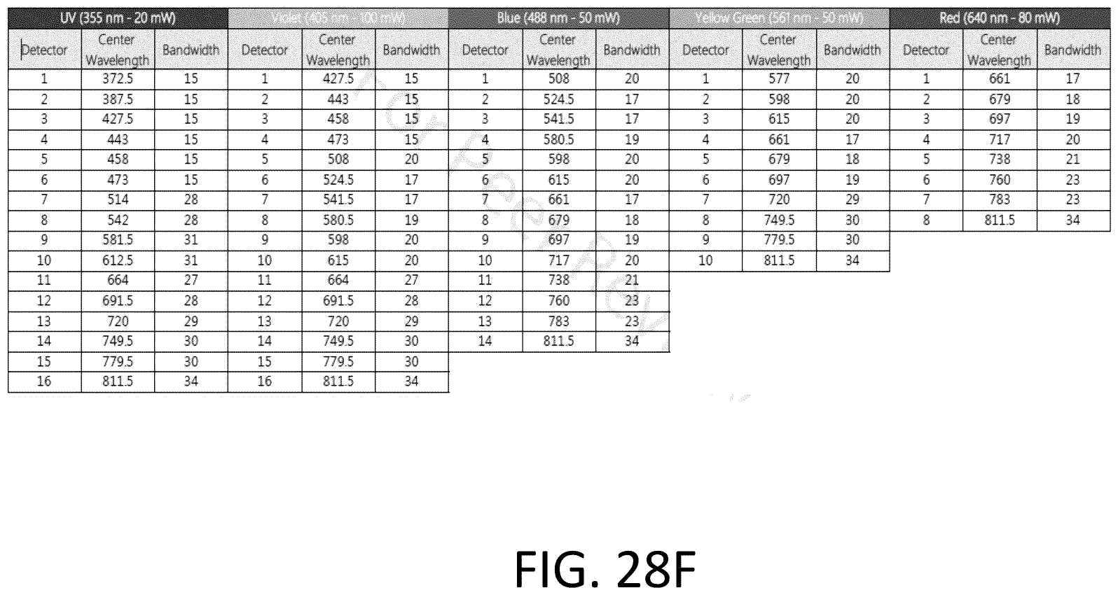

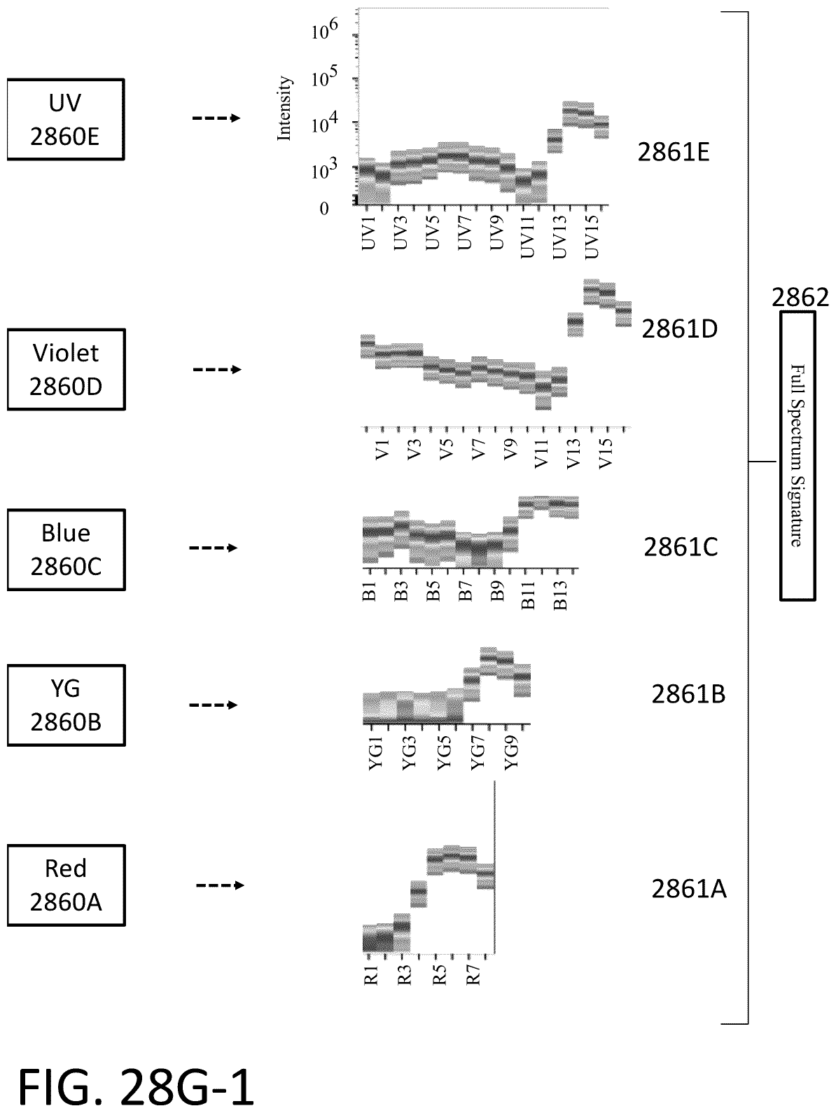

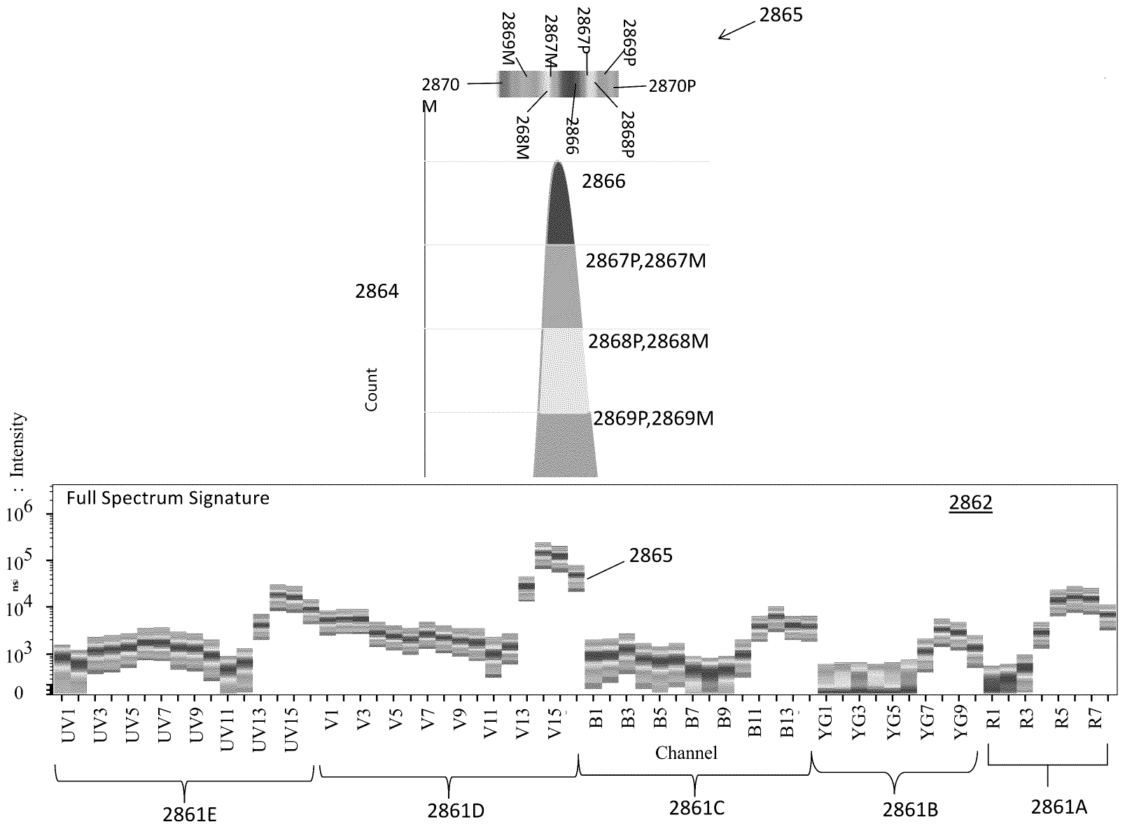

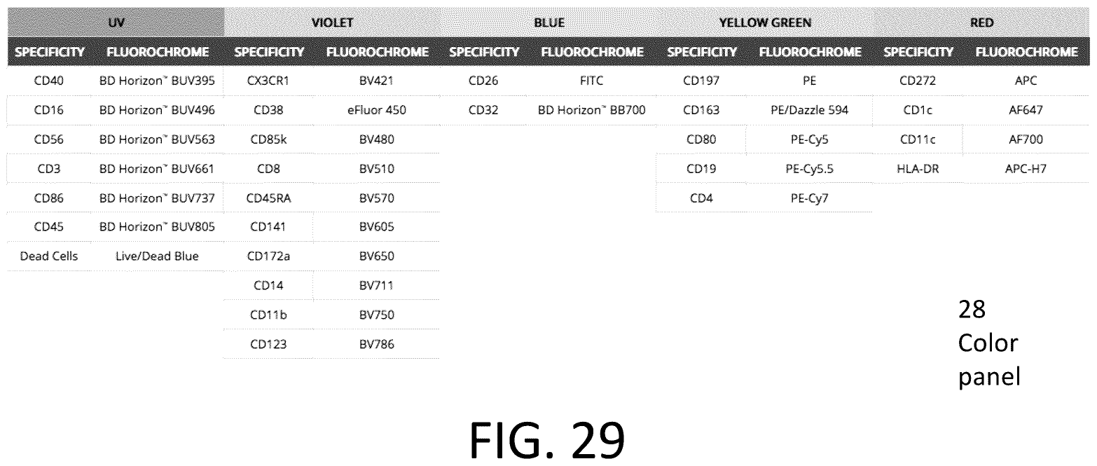

Referring now to FIG. 28A, a flowchart of a method 2800 for a flow cytometer is shown. The flow cytometry system 2500 of FIG. 25, or other flow cytometer systems (e.g., system 2850 shown if FIG. 28E) disclosed herein, can carry out the method 2800. Flow cytometry allows for data collection and analysis of data on single cells or particles of a plurality that are in a sample fluid.

In step 2801, the system starts up the flow cytometer. In step 2802, the system checks the performance of the flow cytometer and performs calibration if and as needed with calibration beads. If the flow cytometer was recently calibrated (e.g., same day or same hour), this step can be skipped.

In step 2803, multiple experiments are setup to run to generate spillover vectors for each dye. A reference sample is prepared (fluorochrome conjugated to an antibody that is attached to a cell or a bead) to initially run to generate event vectors that can be converted into a spillover vector.

In step 2804, the reference sample fluid with one fluorochrome is run through the flow cytometer for analysis with the data captured from N detectors being recorded. Multiple runs through the flow cytometer with the same reference sample fluid may be performed to be sure measurements are well understood. The data from N detectors is recorded for each run of the reference sample through the flow cytometer.

In step 2805, after the sample fluid or calibration beads are run through the flow cytometer, the recorded data can be analyzed to determine results from the analysis by the flow cytometer.

Each spillover vector for one fluorochrome can be subsequently compared with another spillover vector for another fluorochrome to determine how different combinations of pairs of fluorochromes (dyes) and markers interact and spectrally interfere. The spillover vectors for each dye can be subsequently combined together into a spillover matrix for a total number and types of dye being used together to identify cells/particles in a single sample. Combinations of pairs of spillover vectors (columns) in the spillover matrix can be compared together to determine a similarity index between the two fluorochromes. For each reference sample, the light intensity density for each channel can saved as a reference vector and the data can be binned and plotted to form a full spectrum signature for the given fluorochrome.

The flow cytometer can also be shut down if no further samples or calibration beads are to be run. Alternatively, another sample or more calibration beads can be run through the flow cytometer to obtain and record (save) data and subsequently analyze the recorded data.

In step 2805, the system performs single stained compensation controls to generate an initial spillover matrix or reference matrix. When performing multicolor flow cytometry, the system uses single stained samples (reference samples) 2810A-2810E (collectively referred to by reference number 2810) run through a flow cytometer 2500,2850 to determine the levels of compensation, such as shown in FIG. 28B. Single staining of the particles 2810A-2810E can reveal the respective spectral profile or signature 2812A-2812E of respective fluorochromes to the fluorescent photodetectors of the instrument. The information obtained from the single stained particles 2810 can be subsequently used to determine a simplicity index and a complexity index of a set of fluorochromes attached to the particles 2810. The information obtained from the single stained particles 2810 can also be subsequently used to determine a reference full spectrum signature for a fluorochrome useful for unmixing data from a mixed sample labeled with multiple fluorochromes.

The staining of the compensation control usually should be as bright or brighter than the sample. Antibody capture beads can be substituted for cells and one fluorophore conjugated antibody for another, if the fluorescence measured is brighter for the control. The exceptions to this are tandem dyes, which cannot be substituted. Tandem dyes from different vendors or different batches must be treated like separate dyes, and a separate single-stained control should be used for each because the amount of spillover may be different for each of these dyes. Also, the compensation algorithm should be performed with a positive population and a negative population. Whether each individual compensation control contains beads, the cells used in the experiment, or even different cells, the control itself must contain particles with the same level of auto-fluorescence. The entire set of compensation controls may include individual samples of either beads or cells, but the individual samples must have the same carrier particles for the fluorophores. Also, the compensation control uses the same fluorophore as the sample. For example, both green fluorescent protein (GFP) and Fluorescein isothiocyanate (FITC) emit mostly green photons, but have vastly different emission spectra. Accordingly, the system cannot use one of them for the sample and the other for the compensation control. Also, the system must collect enough events to make a statistically significant determination of spillover (e.g., about 5,000 events for both the positive and negative population).

During calibration in a conventional flow cytometer, the system obtains an initial spillover matrix from single stained reference controls. In a conventional flow cytometer, the fluorescence signals (e.g., colors) are separated out into discrete fluorescent bands using a series of edge filters and dichroic mirrors. The system detects (e.g., measures) each individual channel with a photo multiplying tube (PMT). During detection of the fluorescent signals, “spillover” can occur between fluorescent bands, which ideally are completely discrete, such as shown in the combined profile 2826. The system defines the spillover (e.g., spillover 2828 in the combined profile 2826 in FIG. 28C) between the fluorescent bands with a spillover matrix [S].

Alternatively, during calibration in a spectral flow cytometer, the system obtains an initial reference matrix from single stained reference controls 2810. Spectral flow cytometry is a technique based on conventional flow cytometry where a spectrograph and multichannel detector (e.g., charge-coupled device (CCD)) is substituted for the traditional mirrors, optical filters and photomultiplier tubes (PMT) in conventional systems. In the spectral flow cytometer, the side scattered light and fluorescence light is collected and coupled into a spectrograph, either directly or through an optical fiber, where the whole light signal is dispersed and displayed as a high-resolution spectrum on the CCD or coupled into one or more multichannel detectors for detection.

In process step 2804 of FIG. 28A, the sample 2820 shown in FIG. 28C is run through the flow cytometer 2500,2850. The sample 2820 includes a plurality of marked cells or particles 2822A-2822E that flow through each laser beam of each laser and generates fluorescent light and/or scattered light referred to as an event. The fluorescent light and/or scattered light is captured and detected in order to identify the particle and generate counts for the various types of particles in the sample 2820. For each particle in the sample fluid 220 passing by the laser beam(s) and fluorescing light and/or scattering light, the system generates, obtains, and/or records data (e.g., event data) representing the overall spectral profile 2826. For example, fluoresced cells in the sample fluid flowing through the flow cytometer are detected. An event occurs per particle/cell. Each full spectrum detection of a fluoresced cell by the detector modules excited by the lasers is an event. The event data for a particle/cell may be defined according to a measured sample event vector.

In step 2805, the system generates a compensated sample event vector (for conventional flow cytometer) or an unmixed sample event vector (for spectral flow cytometer) to count the number of various types of cells or particles in a sample 2822 to obtain a measure of concentration. Generally as shown in FIG. 28D, an inverse matrix 2834 (determined from the initial spillover matrix and/or the initial reference matrix with fine adjustments) is used on the event data representing the spectral profile 2826 to generate the compensated sample event vector or the unmixed sample event vector representing separate spectral profiles or signatures 2836A-2836E of the various auto-luminescence (generated by the cells or particles themselves) or luminescence given off by the fluorochromes tagged to the various cells 2822A-2822E in the sample 2820. For the conventional flow cytometer, the system calculates the compensated event vector based on the initial spillover matrix and the measured sample event vector. For the spectral flow cytometer, the system calculates the unmixed sample event vector based on the initial reference matrix and the measured sample event vector. Additional steps can be taken to obtain even more accurate results using the initial spillover matrix and a reference matrix.

### Full Spectrum Flow Cytometer

Referring now to FIG. 28E (FIGS. 28E-1 and 28E-2), a schematic diagram of a full spectrum flow cytometer 2850 is shown. United States (US) Patent application Ser. No. 15/659,610 titled COMPACT DETECTION MODULE FOR FLOW CYTOMETERS filed on Jul. 25, 2017 by inventors Ming Yan et al., and U.S. patent application Ser. No. 15/498,397 titled COMPACT MULTI-COLOR FLOW CYTOMETER filed on Apr. 26, 2017 by David Vrane et al. describes further details of flow cytometers and are incorporated herein by reference.

The full spectrum flow cytometer 2850 can be variably configured with different numbers of lasers and different numbers of detector modules. In one embodiment, the full spectrum flow cytometer 2850 can include five lasers (Red 640 nm, Yellow-Green 561 nm, Blue 488 nm, Violet 405 nm, and UV 355 nm) 2851A-2851E and five detector modules 2852A-2852E as shown in FIG. 28E-1 to provide full spectrum analysis. With five detector modules, each of the detector modules (Red, Yellow-Green, Blue, Violet, and UV) 2852A-2852E can be associated with one of the five lasers as shown in FIG. 28E-1. Each of the five lasers generate laser light of five different wavelengths such as ultraviolet (UV) 355 nm, Violet 405 nm, Blue 488 nm, Yellow Green 561 nm, and Red 640 nm. Equipped with five lasers and five detectors, the full spectrum flow cytometer 2850 can be used to develop color panels with 28 or more colors.

The optical paths of the laser light for each of the five lasers (UV 355 nm, Violet 405 nm, Blue 488 nm, Yellow Green 561 nm, and Red 640 nm) is shown in FIG. 28E-1. The lasers are spatially separated, each having an independent optical path to the flow cell 2855. One or more optical components 2854, such as mirrors, lenses, and filters, can be used to direct the laser light of each laser into the flow cell 2855 to strike particles/cells in the sample fluid as they pass by an interrogation region.

After striking a particle in the flow cell 2855, the fluorescent light is collected and directed through a plurality of optical fibers 2857 and one or more optical elements (e.g., lenses) 2858 into each of the individual detector modules 2852A-2852E. Each of the detector modules 2852A-2852E uses a sequential array of a plurality of avalanche photodiodes (APD) as the photodetectors. The full spectrum flow cytometer 2850 can further include a plurality of scatter detectors, including a forward scatter (FSC) detector 2856A near the flow cell, a blue side scatter detector 2856B near the lens/filters for the red detector module, and a violet side scatter detector 2856C near the lens/filters for the blue detector module. The plurality of scatter detectors are typically used to control data capture by the detector modules in the flow cytometer and data storage in a storage device. Each of the detector modules 2852A-2852E can capture a plurality of raw digital data for a given particle/cell as each laser beam of the plurality of lasers strike the same particle. The plurality of raw digital data is captured at slightly different times (laser delay) as the marked particle/cell passes by each laser beam in the flow channel. For example, the yellow/green laser may first strike the particle generating a first set of raw digital data, the violet laser second generating a second set of raw digital data, the blue laser third generating a third set of raw digital data, the red laser fourth generating a fourth set of raw digital data, and the UV laser lastly generating a fifth set of raw digital data for the same particle. If the plurality of lasers are arranged in a different order along the flow channel, the sequential order of generation of raw digital data by the same particle will be different. While an associated detector module is capturing light from its associated lasers, data from detectors in the other detector modules can be ignored. For example, at the time when the red laser strikes the particle/cell, the data from the red detector module is captured while the data from the UV, violet, yellow green, and blue detector modules can be ignored.

With the addition of the UV laser 2851A and having five detector modules providing sixty-four(64) fluorescence detectors (see FIG. 28G), the full spectrum flow cytometer 2850 has the power to take highly multiplexed assays beyond thirty (30) colors. The incorporation of the UV laser 2851A allows the full spectrum flow cytometer 2850 to perform at a different wavelength and discriminate different colors than those systems without. The UV laser enables the use of UV light excited fluorochromes, such as BUV737 and BUV395 fluorochromes, giving researchers additional flexibility on how they design experiments for a sample of particles.

FIG. 28F illustrates the configuration of each photodetector in each of the five detector modules 2852A-2852E used in the embodiments of a full spectrum flow cytometer 2850. Each detector has a bandpass filter in front of it to filter out light. The bandpass filter allows predetermined wavelengths through to the photo detector for detection while filtering out other wavelengths. The detector number (also referred to herein as channel number) and wavelength information of the bandpass filters associated with each photo-detector is shown. The ultraviolet (UV) detector module 2852E has sixteen (16) detectors labeled as channels UV1-UV16 based on their position in the sequential array of detectors in the module. The violet detector module 2852D has sixteen (16) detectors labeled as channels V1-V16 based on their position in the sequential array of detectors in the module. The blue detector module 2852C has fourteen (14) detectors labeled as channels B1-B14 based on their position in the sequential array of detectors in the module. The yellow green detector module 2852B has ten (10) detectors labeled as detector channels YG1-YG10 based on their position in the sequential array of detectors in the module. The red detector module 2852A has eight (8) detectors labeled as detector channels R1-R8 based on their position in the sequential array of detectors in the module.

The multiple lasers in the flow cytometer are slightly spaced apart and sequentially strike the same particle/cell as it flows through the flow channel. This sets up a small amount of time delay between each subsequent laser strike (laser intercept) of the same particle/cell. There is a similar amount of time delay in the respective signal detected by the detectors and the generation of digital data from each laser strike (laser intercept) for the same particle/cell. The small amount of time is referred to as laser delay time and is predetermined by running a quality control experiment (e.g., daily QC runs) before running an experiment with a biological sample or other control. The full spectrum of fluorescence light from each laser striking the particle/cell is sent to each detector module by the fiber optic cables 2857. Based on the laser delay time, the data generated by the detectors from each laser strike (laser intercept) can be associated with a given laser. For example, at one point in time a blue laser strikes the particle/cell and the detectors in the blue detector module can detect fluorescence and generate data for the blue laser strike. After a predetermined laser delay time between blue and red lasers, the same particle is struck by the red laser. Based on the time of the red laser strike, the detectors in the red detector module can detect fluorescence and generate data associated with the red laser strike. The laser delay time between the different lasers can be different but predetermined in order to be able to associate the captured data with the appropriate laser. Furthermore, the arrangement of the lasers can be in a different sequential order such that the sequence of laser strikes can differ. Moreover, the associated laser delay time can differ between laser strikes between power cycles of the flow cytometer. In any case, the data generated by each respective module that is delayed from the first data generated, is aligned together in time and associated with the particle/cell of a single event. The captured data from each detector module may be tagged with a particle/cell number count in the sample run and temporarily stored in a storage device, such as a register, memory or hard drive, for subsequent alignment together as a single event.

Fluorochromes are excited over a wavelength range (excitation wavelength range) associated with the wavelength of the laser and when excited, can emit fluorescence over a different wavelength range (emission wavelength range). The wavelength range of each detector module is associated with the expected emission wavelength range from the excitation of fluorochromes for the associated laser.

With reference to FIG. 28F, the bandpass filter before each detector is used to selectively pass the desirable wavelengths in the pass band range to be detected at a given photo detector for the associated excitation laser. The band bass filter rejects the wavelengths of light outside the pass band range of wavelengths. For example, the first red detector channel (R1 detector channel), the band pass filter has a center wavelength of 661 nanometers (nm) and a bandwidth of 17 nanometers around the center wavelength. Accordingly, in the band pass of wavelengths, a detector can reliably detect a wavelength range around a center wavelength and plus and minus one half the bandwidth. In the case of the R1 detector channel shown in FIG. 28F, the wavelength range is from the center wavelength minus one half the bandwidth (661 nm−8.5 nm=652.5 nm) to the center wavelength plus one half the bandwidth (661 nm+8.5 nm=669.5 nm). In the case of the R8 detector channel, the wavelength range is from the center wavelength minus one half the bandwidth (811.5 nm−17 nm=794.5 nm) to the center wavelength plus one half the bandwidth (811.5 nm+17 nm=828.5 nm). Accordingly, the red detector module detects fluorescent light over a wavelength range from 625 nm to 828.5 nm for fluorescent particles excited by the red laser. The yellow green detector module detects fluorescent light over a wavelength range from 567 nm to 828.5 nm for fluorescent particles excited by the yellow green laser. The blue detector module detects fluorescent light over a wavelength range from 498 nm to 828.5 nm for fluorescent particles excited by the blue laser. The violet detector module detects fluorescent light over a wavelength range from 420 nm to 828.5 nm for fluorescent particles excited by the violet laser. The ultra violet detector module detects fluorescent light over a wavelength range from 365 nm to 828.5 nm for fluorescent particles excited by the ultra violet laser. This detection range includes the full visible light (electromagnetic) spectrum from 380 nm to 780 nm, a portion (365 nm to 379 nm) of the non-visible UV light spectrum, and a portion (781 nm to 828.5 nm) of the non-visible infrared light spectrum.

If even more than 64 detectors are used, an increased granularity in the data at various wavelengths can be captured. The compactness of photo detectors (e.g., avalanche photo-diodes) and the detector array in the detector module has led to embodiments of up to 64 detectors and can lead to a further increase in the numbers of available detectors. A larger number of detectors can lead to increased numbers of colors that can be detected (discriminated) and an increased number of fluorochromes that can be used to examine particles within a single sample by a single run through a flow cytometer. The use of compact photodetectors in a compact photo detector array as the detector modules in the full spectrum flow cytometer 2850 has improved the efficiency of running samples through a flow cytometer and examining the resultant data.

While a single particle has been described passing through each laser, a sample fluid run through a flow cytometer can have thousands of cells/particles per micro liter with hundreds of thousands or more of particles in a sample fluid size of hundreds of microliters (e.g., 500,000 particles in a 500-microliter sample size). The same sample can have different types of cells with hundreds of thousands or more. With a multi-color experiment, different fluorochromes are attached to different particles/cells to count different types of particles in the same sample. In a single run through the flow cytometer, the intensity and wavelength of each color of fluorescent light generated by the excited fluorochrome on the labeled cells can be detected and plotted on a chart by wavelengths to indicate the spectrum of light captured by the sample run. Furthermore, the intensity of fluorescent light for each given color/detector channel can be binned into count ranges with the particle count falling into these ranges being summed up together and plotted on the chart to show the particle cell density for the wavelengths of light.

In FIG. 28E-2, the charts 2860A-2860E of data, normalized intensity (Y axis) versus wavelength (X axis), represents the range of light spectral components captured by each respective detector module for all events (each cell passing through the lasers) in a sample, such as a reference control with a single fluorochrome being used to generate a reference full spectrum signature. In FIG. 28G-1, the raw channel data captured for each detector module 2852A-2852E can respectively be plotted, based on the detector channel number, as a portion (individual detector module spectrum signature) 2861A-2861E of a full spectrum (spectral) signature of the sample run. In the plots of the individual detector module spectrum signature portions 2861A-2861E associated with each color laser 2851A-2851E and associated detector module 2852A-2852E pairing, the intensity (Y axis) and binned density count are plotted as a function of the detector channel number (X axis). Each of the individual detector module spectrum (spectral) signatures is formed out of a channel spectrum signature, such as channel spectrum signature 2865 for the detector module spectrum (spectral) signature 2861D for example.

The channel spectrum signature is plotted based on a plurality of binned intensity levels and the particle counts within those bins. For example, the greatest count (highest density) at the binned intensity level range for the channel is given a first color (e.g., red) located at the center intensity level range 2866 of the channel spectrum signature 2865. For each channel spectrum signature, the other binned intensity levels are either above 2867P,2868P,2869P or below 2867M,2868M,2869M the center intensity level 2866 having the greatest particle/cell count. The second intensity levels 2867P,2867M respectively just above 2867P and below 2867M the center intensity level 2866 are assigned a second color differing from the first color of the center intensity level. The third intensity level 2868P above the second and center intensity levels and the third intensity level 2868M below the second and center intensity levels are assigned a third color differing from the first and second colors. The fourth intensity level 2869P above the third, second, and center intensity levels and the fourth intensity level 2869M below the third, second and center intensity levels are assigned a fourth color differing from the first, second, and third colors. In this manner, intensity density information can be communicated to the user for a given detector channel.

After generating plots of the individual detector module spectrum (spectral) signatures 2861A-2861E, the plots of the individual detector module spectrum (spectral) signatures can then be merged together. In FIG. 28G, the individual detector module spectrum (spectral) signatures 2861A-2861E are merged together along an X axis of detector channel number to form a plot of a full spectrum (spectral) signature 2862 of the exemplary sample run through the full spectrum flow cytometer. Along the X axis, from right to left, are the red detector module spectrum signature 2861A, the yellow green detector module spectrum signature 2861B, the blue-detector module spectrum signature 2861C, the violet detector module spectrum signature 2861D, and the ultraviolet detector module spectrum signature 2861E merged together forming the full spectrum signature for a given sample run. Different labeled samples run through the flow cytometer 2850, will generate different detector module signatures and accordingly different merged full spectrum (spectral) signatures. Single stained control samples (reference controls) are run through the full spectrum flow cytometer used to determine the full spectrum signature of each fluorochrome before being used with other fluorochromes to label a particle/cell in a mixed sample of a plurality of particles/cells.

Instead of just looking at peak intensity levels, the full spectrum signature for one fluorochrome can be used to distinguish from noise and another fluorochrome having a different full spectrum signature. Detecting light intensity over the full spectrum is an advantage of a full spectrum flow cytometer over that of a conventional flow cytometer that just looks at peak intensity levels. When a conventional flow cytometer shows overlap in the spectrum plots of fluorescent dies, the full spectrum signatures of each when run through a full spectrum flow cytometer can be distinguishable. In planning an experiment, it is desirable to select different fluorochromes that can be distinguishable from each other by their full spectrum signatures. Fluorochromes with similar emission but different spectral signatures can be distinguished from each other. The mathematical method to differentiate between multiple fluorophores (mixed fluorescent light) is called spectral unmixing and results in an unmixing matrix that is applied to the captured data of the sample.

Particles/cells may auto-fluoresce (autofluorescence) when struck by the five lasers and have its own full spectrum signature. Accordingly, the autofluorescence of the various particles/cells can also be unmixed, based on the autofluorescence full spectrum signature, and be used to distinguish it from other particle/cell types and the fluorochrome attached to other cells in a mixed sample.

### Optimized Multicolor Immunofluorescence Panel (OMIP)

A 28 color Optimized Multicolor Immunofluorescence Panel (OMIP) is illustrated in FIG. 3. The 28 color OMIP was developed using a full spectrum five laser cytometer as in embodiments of the invention. Markers are listed in the SPECIFICITY columns and corresponding fluorochromes are listed under the FLUOROCHROME columns. Markers and fluorochromes are further grouped under the laser that will optimally excite the fluorochrome.

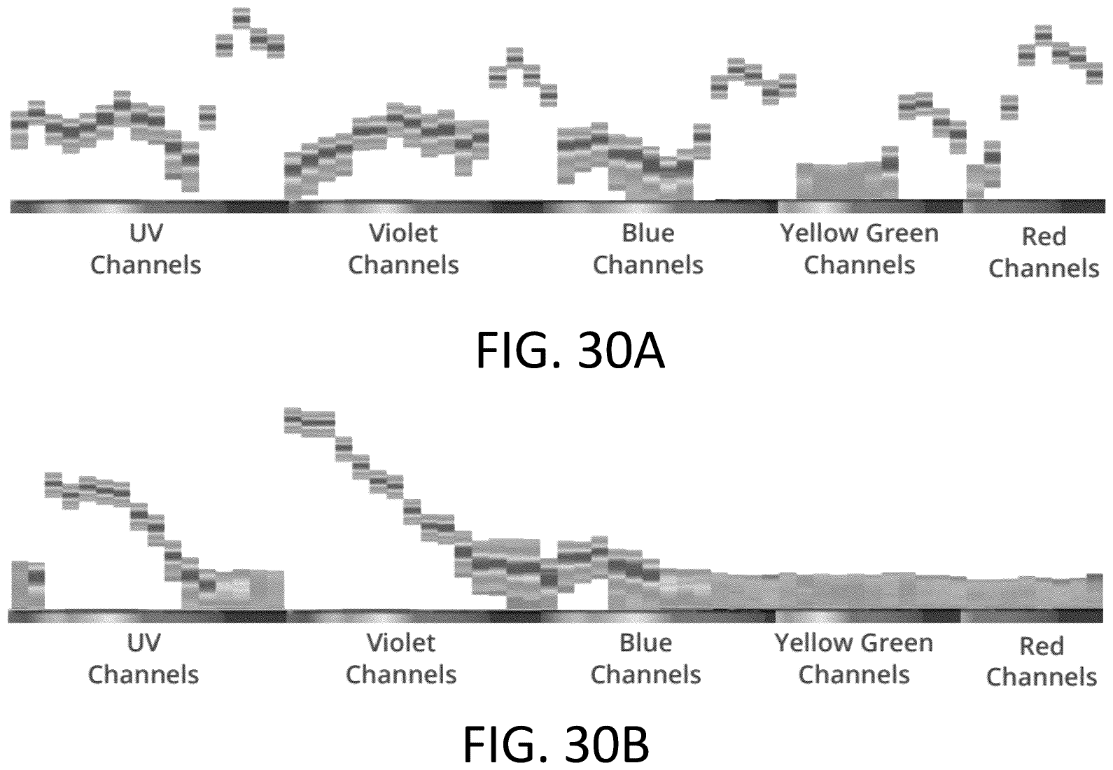

The UV lasers adds an additional 16 fluorescence channels over the full emissions spectra, allowing the invention to extract even more information from each fluorochrome. The spectrum signature of BV737 and BV 421 are shown in FIGS. 30A and 30B respectively. In this example, 16 UV channels gives the BV421 spectrum signature a whole new look. The UV laser allows for a more defined spectrum, allowing for more fluorochromes to be used in the same sample tube minimizing color bleed.

### Configurable Flow Cytometer.

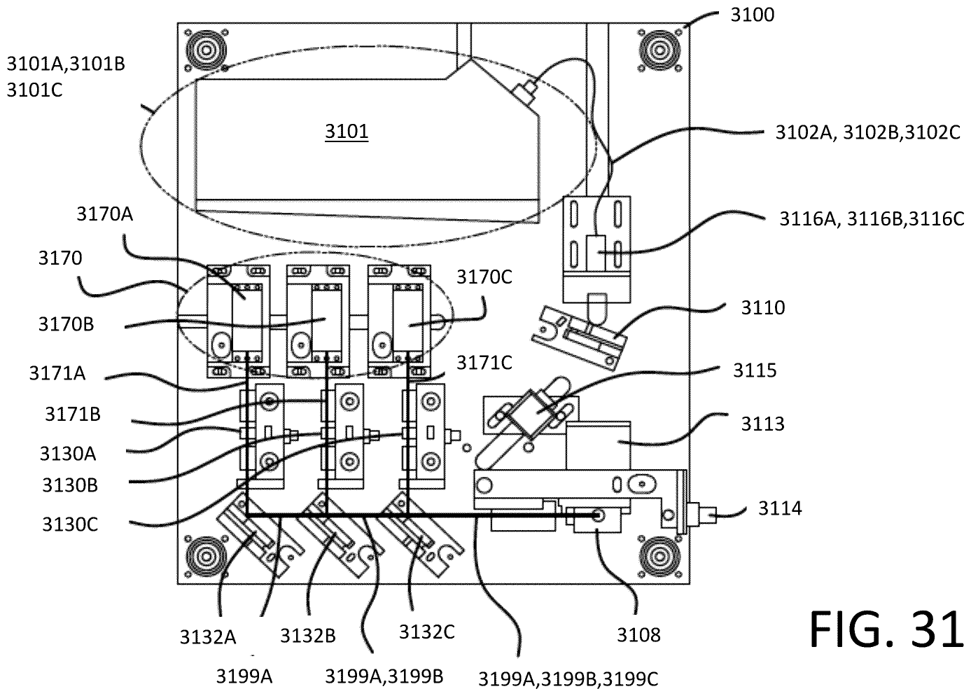

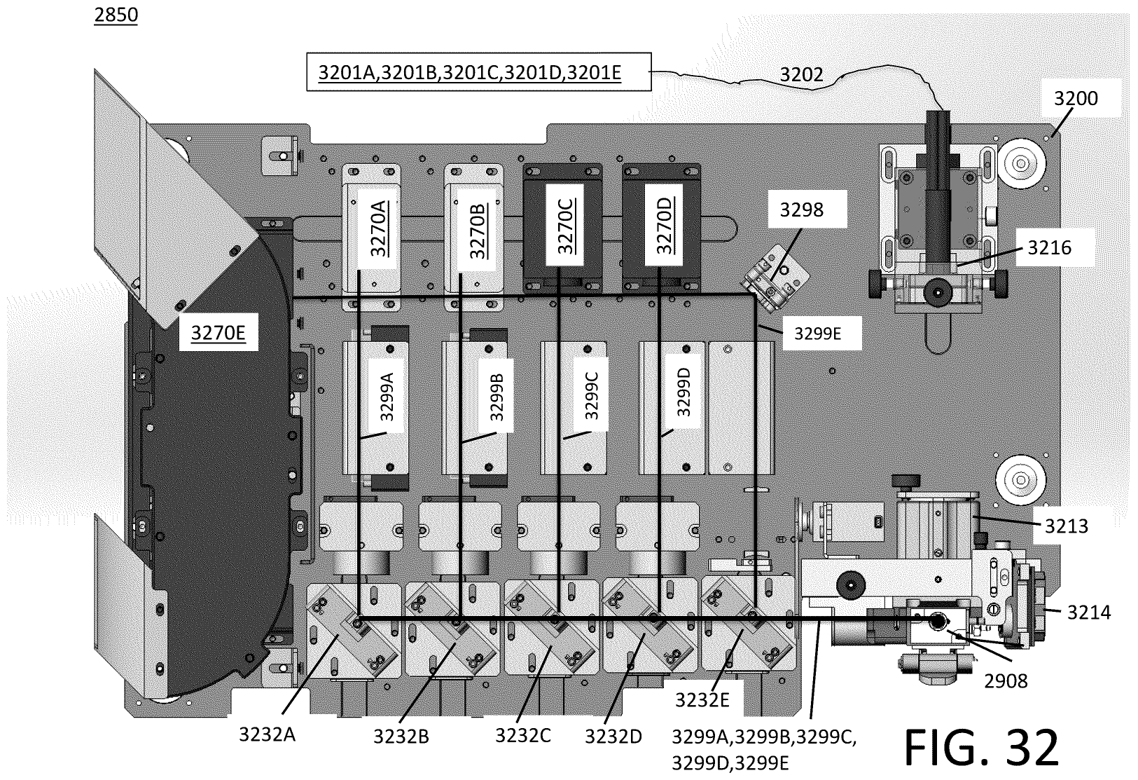

Referring now to FIGS. 31 and 32, a portion of the optical analysis system of modular flow cytometers are shown. The top view of an optical plate assembly 3100,3200 in a modular configurable flow cytometry system is shown. A modular configurable flow cytometer system is configurable in that different combinations of numbers of lasers (e.g., 1, 2, 3, 4, 5) and numbers of detectors (e.g., 14, 16, 22, 30, 32, 38, 48, 54, 64, 128, 256) can be chosen and installed in the flow cytometer. A flow cytometer can be configured with a combination of one, two three, four, five (5) or more lasers and fourteen, sixteen, twenty-two, thirty, thirty-eight, forty-eight, fifty-four, sixty-four (64) or more detectors. With four or more lasers and forty-eight or more detectors, a flow cytometer can act as a full spectrum flow cytometer capturing more electromagnetic spectra than that of a three laser and a thirty-eight-detector configuration.

FIG. 31 shows a top view of an optical plate assembly 3100 for a modular flow cytometry system 100. The optical plate assembly 3100 includes a laser system 3170 having three semiconductor lasers 3170A,3170B,3170C that direct excitation into a flow cell assembly 3108 where a sample fluid flows with sample particles. The laser system 3170 attempts to direct the multiple (e.g., three to five) laser beams in a parallel manner toward the flow cell assembly 3108. The multiple laser beams are slightly offset from one another. The laser system 3170 includes semiconductor lasers 3170A,3170B,3170C. The semiconductor laser generate laser beams having different wavelengths, such as 405 nanometers (nm), 488 nm, and 640 nm for example. The output power of the semiconductor lasers can differ as well. For example, a 405 nm semiconductor laser can generate a laser beam that with an output power that is usually larger than 30 milliwatts (mW). The output power of a 488 nm semiconductor laser is usually greater than 20 mW. The output power of a 640 nm semiconductor laser is usually greater than 20 mW. Controller electronics in the flow cytometer control the semiconductor lasers to operate at a near constant temperature and a near constant output power.

An optical system spatially manipulates the optical laser beams 3171A,3171B,3171C generated by the semiconductor lasers 3170A,3170B,3170C respectively. The optical system includes lenses, prisms, and steering mirrors to focus the optical laser beams onto a fluidic stream carrying biological cells (bio cells). The focused optical laser beam size is typically focused for 50-80 microns (μm) across the flow stream and typically focused for 5-20 μm along the stream flow in the flow cell assembly 3108.

In FIG. 31, the optical system includes beam shapers 3130A-3130C that receive the laser light 3171A,3171B,3171C from the semiconductor lasers 3170A-3170C, respectively. The laser light output from the beam shapers 3130A-3130C are coupled into mirrors 3132A-3132C respectively to direct the laser light 3199A,3199B,3199C towards and into the flow cell assembly 3108 to target particles (e.g., biological cells) stained with a dye of fluorochromes. The laser light 3199A,3199B,3199C is slightly separated from each other but directly substantially in parallel by the mirrors 3132A-3132C into the flow cell assembly 3108.

The laser light beams 3199A,3199B,3199C strike the particles/cells as they pass by in the flow stream in the flow cell assembly 3108. The laser light beams 3199A,3199B,3199C are then scattered by the particles/cells in the flow stream causing the fluorochromes to fluoresce and generate fluorescent light, and the particles/cells to autofluorescence. A forward scatter diode 3114 gathers on-axis scattered light. A collection lens 3113 gathers the off-axis scattered light and the fluorescent light and directs them together to a dichromatic mirror 3110. The dichromatic mirror 3110 focuses the off-axis scattering light onto a side scatter diode 3115. The dichromatic mirror 3110 focuses the fluorescent light onto at least one fiber head 3116. At least one fiber assembly 3102 routes the fluorescent light toward at least one detector module 3101.

For a more detailed analysis of a biological sample using different fluorescent dyes and lasers wavelengths, multiple fiber heads 3116,3216, multiple fiber assemblies 3102,3202 and multiple detector modules 3101,3201 can be used. For example, three or more fiber heads can be used (e.g., see FIG. 31 with three, and FIG. 32 with five) with three or more detector modules associated with three or more lasers.

FIG. 31 shows three fiber heads 3116A,3116B,3116C situated in parallel to receive the fluorescent light and three fiber assemblies 3102A,3102B,3102C can be used to direct the fluorescent light to three detector modules 3101A,3101B,3101C (only one of which is shown in FIG. 31). The first detector module 3101A is located on the optical plate 3100 while the other detector modules are located on a different level. The three fiber heads 3116A,3116B,3116C (and three fiber assemblies 3102A,3102B,3102C) for the three different detector modules paired with the three laser light beams 3199A,3199B,3199C which are slightly offset from each other (e.g., not precisely co-linear). Accordingly, three fiber heads 3116A,3116B,3116C can collect light beam data separately fluorescent light generated by the three laser light beams 3199A,3199B,3199C, having three different wavelengths to excite fluorochromes. The three fiber assemblies 3102A,3102B,3102C then direct light into three different detector modules (e.g., three different detector modules 3101A, 3101B, 3101C), one of which is located on the optical plate 3100 with others located below the optical plate on a lower level of the flow cytometer.

FIG. 32 shows an optical plate 3200 for a full spectrum flow cytometer having a configuration of five lasers and five detector modules with sixty-four photodetectors. The optical plate 3200 has some similar elements to the optical plate 3100. The optical plate 3200 has five fiber heads 3216 for five detector modules (detector modules located off the optical plate). The optical plate 3200 has five lasers 3270A-3270E, one of which is a violet laser 3270D and another one of which is a UV laser 3270E, for exciting and detecting light over the full visible spectrum, including a portion of the UV wavelength spectrum. The laser light beams 3299A,3299B,3299C,3299D are generated in parallel by the lasers 3270A,32070B,32070C,3270D. The UV laser light beam 3299E is generated by the UV laser 3270E spaced apart and initially perpendicular to the laser beams 3299A,3299B,3299C,3299D. The UV laser light beam 3299E is reflected by a first mirror 3298 on the optical plate and directed to run in parallel to the laser beams 3299A-3299D generated by the respective lasers. The mirrors 3232A,3232B,3232C,3232D,3232E respectively receive the laser beams 3299A-3299E along their parallel but different paths, and reflect the laser beams to the flow cell assembly 3208 spaced apart in parallel along the same path.

The optical plate 3200 includes a forward scatter detector 3214 that gathers on-axis scattered light from the particles/cells. A collection lens 3213 coupled to the flow cell assembly 3208 gathers the off-axis scattered light, the fluorescent light, the auto fluorescent light and directs them together to the fiber heads 3216.

The violet and UV lasers and violet and UV detectors differ from the lasers and detectors of the flow cytometer with the optical plate 3100. The violet and UV detector modules have more photodetectors and therefore detect a wider range of wavelengths of fluorescence light when violet and UV lasers strike a particle/cell. With the UV laser 3270E on the optical plate 3200, the detector modules 3201A,3201B,3201C,3201D,3201E (collectively referred to as detector modules 3201) are moved off the optical plate 3200. With a plurality of fiber assemblies 3202 and fiber heads 3216, the light from the flow cell 3208 can be directed into the plurality of different detector modules 3201 in different locations of the flow cytometer.

Not only can the excitation be modular (and configurable) in a modular flow cytometry system, but the detection can also be modular. The modular flow cytometry system can also use one or more detector modules 3101,3201 to collect the light beam data. For example, one or more fiber assemblies can direct light from a flow cell into one or more differing detector modules with different arrays of photodetectors and bandpass filters. For full spectrum signatures, a plurality of (four or more) different detector modules can be used. With the selection of detector modules, the total number of photo detectors (e.g., 16, 32, 64, 128) can differ. The differing detector modules may use different numbers of photodetectors to capture light. Generally, the more detectors one has, the more data can be analyzed and the increased spectral resolution can be achieved.

With a spectral flow cytometer, separation of the light beam data in a mixed sample is handled as a data processing operation over the different detector modules and their respective detectors. The data processing operations can be somewhat complex because separation of the light beam data requires more data manipulation (e.g., identifying different wavelengths and separating light beam data accordingly).

Cell geometric characteristics can be categorized though analysis of the forward and side scattering data. The cells in the fluidic flow are labeled by dyes of visible wavelengths ranging from 400 nm to 900 nm or dyes that fluorescent with ultraviolet non-visible wavelengths when excited by an ultraviolet laser. When excited by lasers, the dyes produce fluorescent light, which are collected by the fiber assembly and routed toward a detector module. The modular flow cytometry system maintains a relatively small size, partly with the optical plate assembly using compact semiconductor lasers in the visible spectrum, a multipower collection lens 3113,3213, and compact image detector arrays in the detector modules. That is, the collection lens 3113,3213 contributes to the design of the compact detector modules.

The collection lens can have a short focal length for the its multipower factor (e.g., 11.5 X power). The collection lens, an objective lens, has a high numerical aperture (NA) facing the fluorescence emissions to capture more photons in the fluorescence emissions over a wide range of incident angles. The collection lens has a low NA of about facing the fibber head and its collection fiber to launch the fluorescent light into the fiber over a narrow cone angle. Accordingly, the collection lens converts from a high NA on one side to a low NA on the opposite side to support a magnification M in the input channel of each detector module.

The diameter of the core of the collection fiber assembly is between about 400 μm and 800 μm, and the fiber NA is about 0.12 for a core diameter of about 600 μm. The fiber output end can be tapered to a core diameter of between about 100 μm and 300 μm for controlling the imaging size onto the receiving photodiode.

The input end of the collection fiber can also include a lensed fiber end to increase the collection NA for allowing use of a fiber core diameter that is less than about 400 μm. Because the collection fiber has the flexibility to deliver the light anywhere in the flow cytometer system, the use of fiber for fluorescence light collection enables optimization of the location of the receiver assembly and electronics for a compact flow cytometer system.

To manufacture a low-cost flow cytometer, lower cost components can be introduced. An image array in each detector module can be formed out of a solid transparent material to provide a detector module that is reliable, low cost, and compact. Furthermore, the flow cytometer can use low cost off the shelf components, such as thin outline (TO) can photodetectors in the detector modules.

The design of a flow cytometer can bring flexibility in selecting fluorochromes for labeling biological cells and particles. Full spectrum cytometry has the advantage of detecting the full spectrum signature for each fluorochrome with a full spectrum flow cytometer with at least five lasers and at least 64 detectors. Almost any commercially available fluorochrome can be excited by the lasers of a full spectrum flow cytometer.

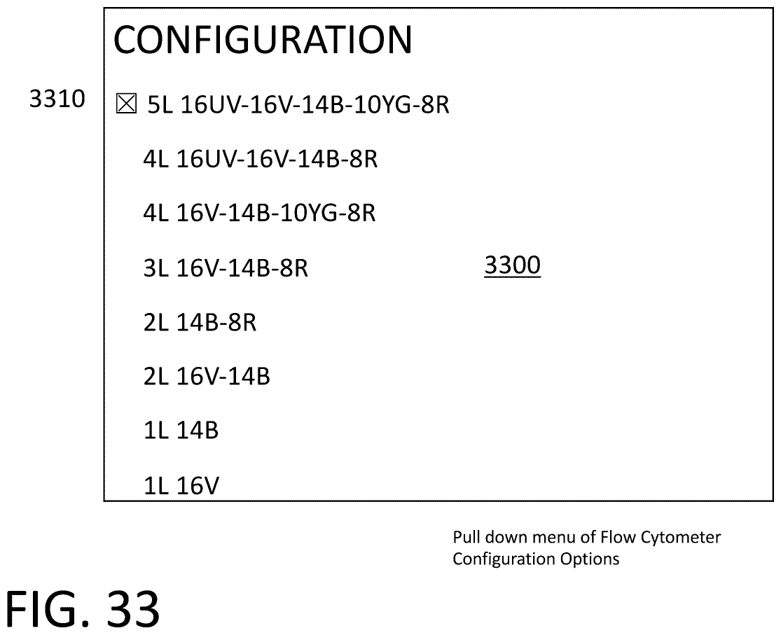

FIG. 33 illustrates a plurality of configurations 3300 that can be selected for forming the modular flow cytometer. A checkmark 3310 illustrates the configuration of 5 L 16UV-16V-15B-10YG-8R. The number in front of L indicates the number of lasers in the modular flow cytometer. For the configuration of 5 L, there are five lasers present in the modular flow cytometer with the different lasers of the different wavelengths. The number in front of the UV indicates the number of ultra-violet detectors over the UV channels of wavelengths. The number in front of the V indicates the number of violet detectors over the violet channels of wavelengths. The number in front of the B indicates the number of blue detectors over the blue channels of wavelengths. The number in front of the YG indicates the number of yellow-green detectors over the yellow-green channels of wavelengths. The number in front of the R indicates the number of red detectors over the red channels of wavelengths.

The emission channels are related to the expected wavelengths of light that the fluorochromes fluoresce. From left to right, the emission channels can include ultraviolet channels UV1-UV16; violet channels V1-V16; blue channels B1-B14; yellow-green channels YG1-YG10; and red channels R1-R8. With fewer lasers and fewer detectors, the channels can decrease. With more lasers and more detectors, the number of channels can increase.

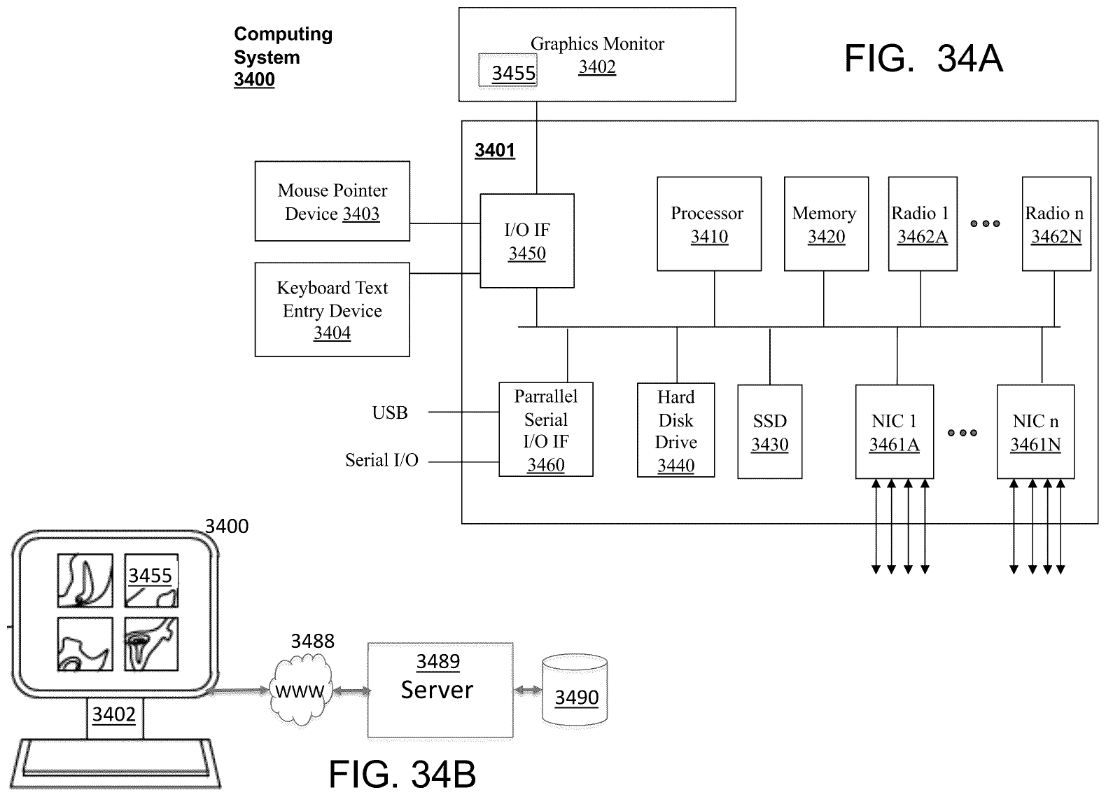

FIG. 34A a block diagram of a computing system 3400 is shown that can execute the software instructions to control the flow cytometer. The computing system 3400 can further execute a web browser to graphically display a graphical user interface (GUI) 3455 to assist a user in selecting fluorochromes that can be used together with the full spectrum flow cytometer in its various configurations. FIG. 34B is a block diagram illustrating the computing system 3400 coupled to a remote computer server 3489 over the cloud or internet 3488. Monitor 3402 illustrates the GUI 3455 generated by the server 3489 and displayed by the computing system 3400. The server 3489 is in communication with a database 3490 that stores information about the available fluorochromes for use with various configurations of a flow cytometer. The information is determined by running each fluorochrome as a reference sample alone through the flow cytometer. A spillover over vector for each fluorochrome is added into a spillover matrix stored in the database 3490. A user can then access the database and select one or more fluorochromes with their underlying data and have graphs charted and the similarity indexes and the complexity index determined.

In one embodiment, the computing system 3400 includes a computer 3401 coupled in communication with a graphics monitor 3402, and one or more input devices, such as a mouse pointer 3403 and a keyboard text entry device 3404. The computer 3401 can couple to other external devices through a plurality of network interfaces 3461A-3461N, a plurality of radio transmitter/receivers (transceivers) 3462A-3462N; and a parallel serial I/O interface 3460.

In accordance with one embodiment, the computer 3401 can include one or more processors 3410, memory 3420; one or more storage drives (e.g., solid state drive, hard disk drive) 3430,840; a video input/output interface 3450A; a parallel/serial input/output data interface 3460; a plurality of network interfaces 3461A-3461N; a plurality of radio transmitter/receivers (transceivers) 3462A-3462N. The graphics monitor 3402 can be coupled in communication with the video input/output interface 3450.

The data interface 3460 can provide wired data connections, such as one or more universal serial bus (USB) interfaces and/or one or more serial input/output interfaces (e.g., RS232). The data interface 3460 can also provide a parallel data interface. The plurality of radio transmitter/receivers (transceivers) 3462A-3462N provide wireless data connections such as over WIFI, Bluetooth, and/or cellular. The one or more audio video devices can use the wireless data connections or the wired data connections to communicate with the computer 3401.

The computer 3401 and computing system 3400 can interface with an external server computer 3489 in the cloud over the internet 3488 through one or more of the plurality of network interfaces 3461A-3461N and/or the plurality of radio transmitter/receivers (transceivers) 3462A-3462N. Each of these network interfaces can support one or more network connections.

One or more computing systems 3400 and/or one or more computers 3401 (or computer servers) can be used to perform some or all of the processes disclosed herein. The software instructions that perform some of the functionality described herein, are stored in the storage device 3430,840 and loaded into memory 3420 when being executed by the processor 3410.

In one embodiment, the processor 3410 executes instructions residing on a machine-readable medium, such as the hard disk drive 3430,840, a removable medium (e.g., a compact disk 3499, a magnetic tape, etc.), or a combination of both. The instructions may be loaded from the machine-readable medium into the memory 3420, which may include Random Access Memory (RAM), dynamic RAM (DRAM), etc. The processor 3410 may retrieve the instructions from the memory 3420 and execute the instructions to perform operations described herein.

The embodiments of the invention are thus described. While embodiments of the invention have been particularly described, they should not be construed as limited by such embodiments, but rather construed according to the claims that follow below.

While certain exemplary embodiments have been described and shown in the accompanying drawings, it is to be understood that such embodiments are merely illustrative of and not restrictive on the disclosed embodiments, and that the disclosed embodiments not be limited to the specific constructions and arrangements shown and described, since various other modifications may occur to those ordinarily skilled in the art.

While this specification includes many specifics, these should not be construed as limitations on the scope of the disclosure or of what may be claimed, but rather as descriptions of features specific to particular implementations of the disclosure. Certain features that are described in this specification in the context of separate implementations may also be implemented in combination in a single implementation. Conversely, various features that are described in the context of a single implementation may also be implemented in multiple implementations, separately or in sub-combination. Moreover, although features may be described above as acting in certain combinations and even initially claimed as such, one or more features from a claimed combination may in some cases be excised from the combination, and the claimed combination may be directed to a sub-combination or variations of a sub-combination. Accordingly, the claimed invention is limited only by patented claims that follow below.

1. A method for producing conjugated inorganic nanoparticle fluorescent dye complexes for flow cytometry and biological applications, the method comprising:
a) covering a surface of a plurality of inorganic nanoparticles with a functional group R using a coupling agent to form a plurality of functionalized inorganic nanoparticles;
b) coupling fluorescent dyes to the plurality of functionalized inorganic nanoparticles to form a plurality of functionalized fluorescent inorganic nanoparticles;
c) coupling a plurality of first linker molecules, attached to a functional group R″, to the plurality of fluorescent functionalized inorganic nanoparticles; and
d) bioconjugating the plurality of fluorescent functionalized inorganic nanoparticles with antibodies or other bioactive molecules.

2. The method of claim 1, wherein using the coupling agent to cover the surface of the plurality of inorganic nanoparticles with functional groups further comprises, hydrolyzing the coupling agent.

3. The method of claim 1, wherein
coupling fluorescent dyes to the functionalized inorganic nanoparticles further comprises, mixing fluorescent dyes attached to the functional group R with functionalized inorganic nanoparticles.

4. The method of claim 1, wherein
coupling a plurality of first linker molecules to the surface of the plurality of functionalized inorganic nanoparticles further comprises, mixing the plurality of first linker molecules, attached to a functional group R′ and a functional group R″, with the functionalized inorganic nanoparticles, wherein the functional group R′ reacts with the functional group R covering the surface of a plurality of inorganic nanoparticles.

5. The method of claim 1, further comprises,
changing the functional group R″, attached to the plurality of first linker molecules, to a functional group R2, by mixing a plurality of second linker molecules to the fluorescent functionalized inorganic nanoparticles,
wherein a functional group of the plurality of second linker molecules reacts with the functional group R″ of the plurality of first linker molecules to form the functional group R2.

6. The method of claim 1, further comprising:
reacting linker molecules or oligomers with a plurality of antibodies or other bioactive molecules to activate the plurality of antibodies or other bioactive molecules.

7. The method of claim 6, further comprising:
directly bio-conjugating functionalized fluorescent inorganic nanoparticles with activated antibodies or other bioactive molecules.

8. The method of claim 1, wherein:
the plurality of inorganic nanoparticles are covered with an inorganic metal oxide.

9. The method of claim 8, wherein:
the plurality of inorganic nanoparticles include alumina nanoparticles, silica nanoparticles, titania nanoparticles, indium tin oxide nanoparticles, zinc oxide nanoparticles, iron oxide nanoparticles, antimony tin oxide nanoparticles, or nanoparticles covered with an inorganic metal oxide layer.

10. The method of claim 1, wherein:
a size of the plurality of inorganic nanoparticles is less than 500 nanometers, 200 nanometers, 100 nanometers, 50 nanometers, 25 nanometers, 15 nanometers, 10 nanometers, or 5 nanometers.

11. The method of claim 1, wherein:
the coupling agent is one of a silane coupling agent, a titanate coupling agent, an aluminate coupling agent, a zirconate coupling agent, a phosphate coupling agent, and a borate coupling agent.

12. The method of claim 1, wherein:
the functional group R on the coupling agent includes one or more of alkylhalide, azide, amino, alkyne, aldehyde, maleimide, hydroxyl, acetal, isocyanate, epoxide, acrylate, sulfonate (tosyl, mesyl), nitrophenyl carbonate, Biotins, folic acid, methacrylate, mercapto, tetrafluorophenyl esters, succinimidyl ester, pentafluorophenyl ester, hydrazides, vinyl, vinylsulfone, dibenzocyclooctyne group (DBCO), and methyltetrazine.

13. The method of claim 1, wherein:
the coupling agent comprises a silicon atom; and
the plurality of first linker molecules that connect the functional group R″ to the silicon atom includes one of an alkyl chain, a peptide chain, and a polyethylene oxide chain.

14. The method of claim 1, wherein:
a number of repeated units of the plurality of first linker molecules ranges from 5,000 to 1, from 3,000 to 1, from 2,000 to 1, from 1,000 to 1, from 500 to 1, from 100 to 1, or from 20 to 1.

15. The method of claim 1, wherein:
the fluorescent dyes is a fluorescent chemical compound that can emit light upon laser excitation.

16. The method of claim 12, wherein:
the fluorescent dyes is at least one of BODIPY derivatives, dipyrrin-metal derivatives, Atto derivatives, Cyanine derivatives, squaraine derivatives, Fluorescein derivatives, porphyrin, metalloporphyrin derivatives, phthalocyanine derivatives, Rhodamine derivatives, lanthanide complexes derivatives, and Pyrene dyes.

17. The method of claim 12, wherein:
the fluorescent dyes is an organic fluorescent dye with a narrow bandwidth of light absorption between 260 nanometers and 900 nanometers and a narrow bandwidth of fluorescence between 260 nanometers and 1100 nanometers.

18. The method of claim 1, wherein:
the fluorescent dyes has functional groups that can react with functional groups on a surface of inorganic nanoparticles.

19. The method of claim 18, wherein:
the functional groups on the fluorescent dyes is at least one of an amino, an alkylhalide, an azide, an alkyne, an aldehyde, a maleimide, a hydroxyl, an acetal, an isocyanate, an epoxide, an acrylate, a sulfonate (tosyl, mesyl), a nitrophenyl carbonate, a Biotins, a folic acid, a methacrylate, a mercapto, a tetrafluorophenyl ester, a succinimidyl ester, a pentafluorophenyl ester, a hydrazides, a vinyl, a vinylsulfone, a dibenzocyclooctyne group (DBCO), and a methyltetrazine and other reactive functional groups.

20. The method of claim 18, wherein:
the functional groups on the fluorescent dyes are reactive functional groups.

21. The method of claim 1, wherein:
the fluorescent dyes react with functional groups on surface of inorganic nanoparticle by a condensation reaction, a click chemistry reaction, a photochemistry reaction, a Suzuki coupling reaction, a Stille coupling reaction, a Sonogashira coupling reaction; or a Heck, Mcmurray and Knoevenagel, Wittig, Horner reaction.

22. The method of claim 1, wherein:
the plurality of first linker molecules with functional groups are an oligomer chain with one functional group, an oligomer chain with two functional groups, or branched oligomers with multi-functional groups.

23. The method of claim 1, wherein:
a functional group R′ and the functional group R″ in the plurality of first linker molecules include one or more of an amino, an alkylhalide, an azide, an alkyne, an aldehyde, a maleimide, a hydroxyl, an acetal, an isocyanate, an epoxide, an acrylate, a sulfonate (tosyl, mesyl), a nitrophenyl carbonate, a Biotins, a folic acid, a methacrylate, a mercapto, a tetrafluorophenyl ester, a succinimidyl ester, a pentafluorophenyl ester, a hydrazides, a vinyl, a vinylsulfone, a dibenzocyclooctyne group (DBCO), an a methyltetrazine.

24. The method of claim 1, wherein:
a functional group R′ and the functional group R″ in the plurality of the first linker molecules are reactive functional groups.

25. The method of claim 1, wherein:
a backbone of the plurality of the first linker molecules are one of an alkyl chain, a peptide chain, and a polyethylene oxide chain.

26. The method of claim 25, wherein:
a number of repeated units in the plurality of the first linker molecules range from 10,000 to 1, from 5,000 to 1, from 3,000 to 1, from 2,000 to 1, from 1,000 to 1, from 500 to 1, from 100 to 1, or from 20 to 1.

27. (canceled)
